(19) United States
(12) Patent Application Publication
(10) Pub. No.: US 2023/0165979 A1
(43) Pub. Date: Jun. 1, 2023

(54) COPPER-CONTAINING THERAGNOSTIC COMPOSITIONS AND METHODS OF USE

(71) Applicants: Cornell University, Ithaca, NY (US); The University of Melbourne, Parkville, Victoria (AU)

(72) Inventors: John W. BABICH, New York, NY (US); James M. Kelly, New York, NY (US); Alejandro Amor-Coarasa, New York, NY (US); Shashikanth Ponnala, New York, NY (US); Paul Donnelly, Parkville, Victoria (AU)

(73) Assignees: Cornell University, Ithaca, NY (US); The University of Melbourne, Parkville, Victoria (AU)

(21) Appl. No.: 17/922,583

(22) PCT Filed: Apr. 14, 2021
(86) PCT No.: PCT/US2021/027276

§ 371 Date: Oct. 31, 2022

Related U.S. Application Data
(60) Provisional application No. 63/020,838, filed on May 6, 2020.

(51) Int. Cl. A61K 51/04; A61K 31/395

(52) U.S. Cl. CPC A61K 51/041; A61K 31/5545

(57) ABSTRACT

The present technology provides compounds, as well as compositions including such compounds, useful for imaging and/or treatment of a non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and/or a metastatic cancer. The compounds include a tumor targeting domain (that includes a moiety capable of recognizing or interacting with a molecular target on the surface of tumor cells), a blood-protein binding domain, and a sarcophagine-containing domain, where the moiety of the tumor targeting domain is distal to and sterically unimpeded by the blood-protein binding domain.

### CROSS-REFERENCE TO RELATED APPLICATIONS

This application claims the benefit of U.S. Provisional Patent Application No. 63/020,838, filed on May 6, 2020, the entire disclosure of which is incorporated herein by reference for any and all purposes.

### FIELD

The present technology generally relates to trifunctional constructs that include a tumor targeting domain (where the tumor targeting domain includes a moiety capable of recognizing or interacting with a molecular target on the surface of tumor cells), a blood-protein binding domain, and a sarcophagine-containing domain, where the moiety of the tumor targeting domain is distal to and sterically unimpeded by the blood-protein binding domain. The sarcophagine-containing domain of the compounds of the present technology is capable of chelating ⁶⁴Cu⁺² or ⁶⁷Cu⁺². The present technology also provides compositions including such compounds as well as methods of use in imaging and/or anti-tumor therapy. For example, the compounds and compositions of the present technology are useful theranostic compounds.

### SUMMARY

In an aspect, a compound is provided that includes a tumor targeting domain (where the tumor targeting domain includes a moiety capable of recognizing or interacting with a molecular target on the surface of tumor cells), a blood-protein binding domain, and a sarcophagine-containing domain, where the moiety of the tumor targeting domain is distal to and sterically unimpeded by the blood-protein binding domain. The tumor targeting domain of embodiment herein may be capable of binding to a tumor associated molecular target that includes one or more of: a tumor associated molecular target that is a tumor-specific cell surface protein or other marker such as prostate specific membrane antigen (PSMA), somatostatin peptide receptor-2 (SSTR2), alphavbeta3 (αvβ3), alphavbeta6, a gastrin-releasing peptide receptor, a seprase (e.g., fibroblast activation protein alpha (FAP-alpha)), an incretin receptor, a glucose-dependent insulinotropic polypeptide receptor, VIP-1, NPY, a folate receptor, LHRH, a neuronal transporter (e.g., noradrenaline transporter (NET)), EGFR, HER-2, VGFR, MUC-1, CEA, MUC-4, ED2, TF-antigen, an endothelial specific marker, neuropeptide Y, uPAR, TAG-72, a claudin, a CCK analog, VIP, bombesin, VEGFR, a tumor-specific cell surface protein, GLP-1, CXCR4, Hepsin, TMPRSS2, a caspace, cMET, or an overexpressed peptide receptor. The tumor targeting domain of any embodiment disclosed herein may include a modified antibody, modified antibody fragment, a modified binding peptide, a prostate specific membrane antigen (“PSMA”) binding peptide, a somatostatin receptor agonist, a bombesin receptor agonist, a seprase binding compound, or binding fragment of any one or more thereof.

In any embodiment disclosed herein of the present technology, the compound may be of any one of Formulas I-V:

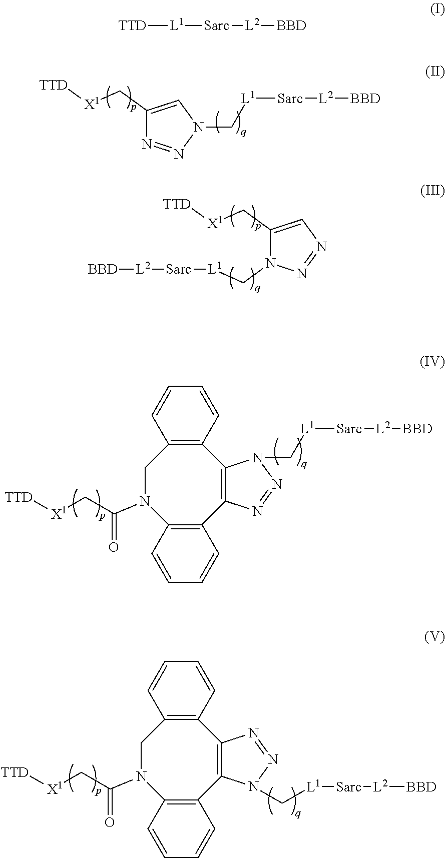

or a pharmaceutically acceptable salt and/or solvate thereof, where
TTD is the tumor targeting domain of any embodiment disclosed herein;
BBD is the blood-protein binding domain of any embodiment disclosed herein;
Sarc is the sarcophagine-containing domain of any embodiment disclosed herein;
X¹ is independently at each occurrence absent, O, S, NH, —C(O)—, —C(O)—NR¹—, —NR²—C(O)—, —C(O)—NR³—C₁-C₁₂ alkylene-, —C₁-C₁₂ alkylene-C(O)—, —C(O)—NR⁴—C₁-C₁₂ alkylene-C(O)—, -arylene-, -heterocyclene-, —O(CH₂CH₂O)ₐ—, —CH₂CH₂—O(CH₂CH₂O)_{b}—, —CH₂CH₂—O(CH₂CH₂O)_{c}—CH₂CH₂—, —O(CH₂CH₂O)_{d}—CH₂CH₂—, —C(O)—O(CH₂CH₂O)ₑ—, —O(CH₂CH₂O)_{f}—CH₂CH₂C(O)—, —C(O)—O(CH₂CH₂O)_{g}—, —C(O)—O(CH₂CH₂O)ₕ—CH₂CH₂—, —C(O)—O(CH₂CH₂O)ᵢ—CH₂CH₂C(O)—, —CH₂CH₂—O(CH₂CH₂O)ⱼ—CH₂CH₂C(O)—, —C(O)—NR—CH₂CH₂O(CH₂CH₂O)ₖ—, —C(O)—NR⁶—CH₂CH₂O(CH₂CH₂O)ₗ—CH₂CH₂—, —C(O)—NR—CH₂CH₂O(CH₂CH₂O)ₘ—CH₂CH₂C(O)—, an amino acid, a peptide of 2, 3, 4, 5, 6, 7, 8, 9, or 10 amino acids, or a combination of any two or more thereof, where a, b, c, d, e, f, g, h, i, j, k, l, and m are independently at each occurrence 0, 1, 2, 3, 4, 5, 6, 7, 8, 9, 10, 11, 12, 13, 14, 15, 16, 17, 18, or 19, and where R¹, R², R³, R⁴, R⁵, R⁶, and R⁷ are independently at each occurrence H, alkyl, or aryl;
L¹ is independently at each occurrence absent, O, S, NH, —C(O)—, —C(O)—NR⁸—, —NR⁹—C(O)—, —C(O)—NR¹⁰—C₁-C₁₂ alkylene-, —C₁-C₁₂ alkylene-C(O)—, —C(O)—NR¹¹—C₁-C₁₂ alkylene-C(O)—, -arylene-, -heterocyclene-, —O(CH₂CH₂O)_{a′}—, —CH₂CH₂—O(CH₂CH₂O)_{b′}—, —CH₂CH₂—O(CH₂CH₂O)_{c′}—CH₂CH₂—, —O(CH₂CH₂O)_{d′}—CH₂CH₂—, —C(O)—O(CH₂CH₂O)_{e′}—, —O(CH₂CH₂O)_{f′}—CH₂CH₂C(O)—, —C(O)—O(CH₂CH₂O)_{g′}—, —C(O)—O(CH₂CH₂O)_{h′}—CH₂CH₂—, —C(O)—O(CH₂CH₂O)_{i′}—CH₂CH₂C(O)—, —CH₂CH₂—O(CH₂CH₂O)_{j′}—CH₂CH₂C(O)—, —C(O)—NR¹²—CH₂CH₂O(CH₂CH₂O)_{k′}—, —C(O)—NR¹³—CH₂CH₂O(CH₂CH₂O)_{l′}—CH₂CH₂—, —C(O)—NR¹⁴—CH₂CH₂O(CH₂CH₂O)_{m′}—CH₂CH₂C(O)—, an amino acid, a peptide of 2, 3, 4, 5, 6, 7, 8, 9, or 10 amino acids, or a combination of any two or more thereof, where a′, b′, c′, d′, e′, f′, g′, h′, i′, j′, k′, l′, and m′ are independently at each occurrence 0, 1, 2, 3, 4, 5, 6, 7, 8, 9, 10, 11, 12, 13, 14, 15, 16, 17, 18, or 19, and where R⁸, R⁹, R¹⁰, R¹¹, R¹², R¹³, and R¹⁴ are independently at each occurrence H, alkyl, or aryl;
L² is independently at each occurrence absent, O, S, NH, —C(O)—, —C(O)—NR¹⁵—, —NR¹⁶—C(O)—, —C(O)—NR¹⁷—C₁-C₁₂ alkylene-, —C₁-C₁₂ alkylene-C(O)—, —C(O)—NR¹⁸—C₁-C₁₂ alkylene-C(O)—, -arylene-, -heterocyclene-, —O(CH₂CH₂O)_{a″}—, —CH₂CH₂—O(CH₂CH₂O)_{b″}—, —CH₂CH₂—O(CH₂CH₂O)_{c″}—CH₂CH₂—, —O(CH₂CH₂O)_{d″}—CH₂CH₂—, —C(O)—O(CH₂CH₂O)_{e″}—, —O(CH₂CH₂O)_{f″}—CH₂CH₂C(O)—, —C(O)—O(CH₂CH₂O)_{g″}—, —C(O)—O(CH₂CH₂O)_{h″}—CH₂CH₂—, —C(O)—O(CH₂CH₂O)_{i″}—CH₂CH₂C(O)—, —CH₂CH₂—O(CH₂CH₂O)_{j″}—CH₂CH₂C(O)—, —C(O)—NR¹⁹—CH₂CH₂O(CH₂CH₂O)_{k″}—, —C(O)—NR²⁰—CH₂CH₂O(CH₂CH₂O)_{l″}—CH₂CH₂—, —C(O)—NR²¹—CH₂CH₂O(CH₂CH₂O)_{m″}—CH₂CH₂C(O)—, an amino acid, a peptide of 2, 3, 4, 5, 6, 7, 8, 9, or 10 amino acids, or a combination of any two or more thereof, where a″, b″, c″, d″, e″, f″, g″, h″, i″, j″, k″, l″, and m″ are independently at each occurrence 0, 1, 2, 3, 4, 5, 6, 7, 8, 9, 10, 11, 12, 13, 14, 15, 16, 17, 18, or 19, and where R, R¹⁶, R¹⁷, R¹⁸, R¹⁹, R²⁰, and R²¹ are independently at each occurrence H, alkyl, or aryl;
p is independently at each occurrence 0, 1, 2, 3, 4, or 5; and
q is independently at each occurrence 1 or 2.

In any embodiment disclosed herein of the compound, the sarcophagine-containing domain may or may not chelate ⁶⁴Cu⁺² or ⁶⁷Cu⁺².

In an aspect, a composition is provided that includes a compound of any embodiment disclosed herein and also includes a pharmaceutically acceptable carrier.

In an aspect, a pharmaceutical composition is provided, where the composition includes an effective amount of a compound of any embodiment disclosed herein that chelates ⁶⁴Cu⁺² or ⁶⁷Cu⁺² for imaging and/or detecting one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer; and a pharmaceutically acceptable carrier. In a related aspect, a method is provided where the method includes administering to a subject an effective amount of a compound of any embodiment disclosed herein that chelates ⁶⁴Cu⁺² or ⁶⁷Cu⁺² for imaging and/or detecting a cancer; and subsequent to the administering, detecting one or more of positron emission, gamma rays from positron emission and annihilation, and Cerenkov radiation due to positron emission.

In an aspect, a pharmaceutical composition is provided, where the composition an effective amount of a compound of any embodiment disclosed herein that chelates ⁶⁴Cu⁺² or ⁶⁷Cu⁺² for treating one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer; and a pharmaceutically acceptable carrier. In a related aspect, a method is provided that includes administering to a subject an effective amount of a compound of any embodiment disclosed herein that chelates ⁶⁴Cu⁺² or ⁶⁷Cu⁺² for treating a cancer.

### BRIEF DESCRIPTION OF THE DRAWINGS

FIGS. 1A-1B shows HPLC chromatograms of spiked samples of the present technology, according to the working examples, where FIG. 1A is of the [⁶⁴Cu]Cu-RPS-085 sample and FIG. 1B is of the [⁶⁷Cu]Cu-RPS-085 sample, where in each the top chromatogram is the UV absorbance at 280 nm and the bottom is the corresponding radiochromatogram.

FIGS. 2A-2B show RadioHPLC chromatograms of [⁶⁷Cu]Cu-RPS-063 after 20 min at 25° C. (FIG. 2A) and following purification by solid phase extraction (FIG. 2B), according to the working examples.

FIG. 3 shows microPET/CT images of [⁶⁴Cu]Cu-RPS-085 distribution in male Balb/C nu/nu mice bearing LNCaP xenograft tumors, according to the working examples.

FIG. 4 illustrates the tissue biodistribution of [⁶⁴Cu]Cu-RPS-085 in male Balb/C nu/nu mice bearing LNCaP xenografts, according to the working examples.

FIG. 5 shows the tissue biodistribution of [⁶⁷Cu]Cu-RPS-085 in male Balb/C nu/nu mice bearing LNCaP xenografts, according to the working examples.

FIG. 6 shows the Time-Activity curves of [⁶⁷Cu]Cu-RPS-085 and [¹⁷⁷Lu]Lu-RPS-063 in LNCaP tumors and the kidneys of male Balb/C nu/nu mice, according to the working examples.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated.

No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 weight %” would be understood to mean “9 weight % to 11 weight %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, or B and C.”

As used herein, the term “amino acid” includes naturally-occurring α-amino acids and synthetic α-amino acids (e.g., 2-amino-2-phenylacetic acid, also referred to as phenylglycine), as well as α-amino acid analogues and amino acid mimetics that function in a manner similar to the naturally-occurring amino acids. The term further includes both L and D forms of such α-amino acids unless a specific stereoisomer is indicated. Naturally-occurring amino acids are those encoded by the genetic code, as well as those amino acids that are later modified, e.g., hydroxyproline, γ-carboxyglutamate, and O-phosphoserine. Amino acid analogues refer to compounds that have the same basic chemical structure as a naturally-occurring amino acid, e.g., an α-carbon bearing an organic group, e.g., homoserine, norleucine, methionine sulfoxide, methionine methyl sulfonium. Such analogues may have modified organic groups (e.g., norleucine) or modified peptide backbones, but retain the same basic chemical structure as a naturally-occurring amino acid. Amino acid mimetics refer to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art, as well as synthetic amino acids.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include, but are not limited to: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF5), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; and nitriles (i.e., CN).

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

As used herein, Cₘ-Cₙ, such as C₁-C₁₂, C₁-C₈, or C₁-C₆ when used before a group refers to that group containing m to n carbon atoms.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Alkyl groups may be substituted or unsubstituted. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, and carboxyalkyl.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Cycloalkyl groups may be substituted or unsubstituted.

Substituted cycloalkyl groups may be substituted one or more times with, non-hydrogen and non-carbon groups as defined above. However, substituted cycloalkyl groups also include rings that are substituted with straight or branched chain alkyl groups as defined above.

Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Cycloalkylalkyl groups may be substituted or unsubstituted. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Alkenyl groups may be substituted or unsubstituted. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above.

Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C¢CH, —C≡CCH₃, —CH₂C≡CCH₃, —C≡CCH₂CH(CH₂CH₃)₂, among others. Alkynyl groups may be substituted or unsubstituted. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Aryl groups may be substituted or unsubstituted. The phrase “aryl groups” includes groups containing fused rings, such as fused aromatic-aliphatic ring systems (e.g., indanyl, tetrahydronaphthyl, and the like). Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Aralkyl groups may be substituted or unsubstituted. Substituted aralkyl groups may be substituted at the alkyl, the aryl or both the alkyl and aryl portions of the group.

Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl.

Heterocyclyl groups may be substituted or unsubstituted. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Heteroaryl groups may be substituted or unsubstituted. Thus, the phrase “heteroaryl groups” includes fused ring compounds as well as includes heteroaryl groups that have other groups bonded to one of the ring members, such as alkyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above.

Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heterocyclyl groups are heterocyclene groups, divalent heteroaryl groups are heteroarylene groups, and so forth. Substituted groups having a single point of attachment to the compound of the present technology are not referred to using the “ene” designation. Thus, e.g., chloroethyl is not referred to herein as chloroethylene. Such groups may further be substituted or unsubstituted.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like.

Examples of cycloalkoxy groups include but are not limited to cyclopropyloxy, cyclobutyloxy, cyclopentyloxy, cyclohexyloxy, and the like. Alkoxy groups may be substituted or unsubstituted. Representative substituted alkoxy groups may be substituted one or more times with substituents such as those listed above.

The terms “alkanoyl” and “alkanoyloxy” as used herein can refer, respectively, to —C(O)-alkyl and —O—C(O)-alkyl groups, where in some embodiments the alkanoyl or alkanoyloxy groups each contain 2-5 carbon atoms. Similarly, the terms “aryloyl” and “aryloyloxy” respectively refer to —C(O)-aryl and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylic acid” as used herein refers to a compound with a —C(O)OH group. The term “carboxylate” as used herein refers to a —C(O)O— group. A “protected carboxylate” refers to a —C(O)O-G where G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “ester” as used herein refers to —COOR⁷⁰ groups. R⁷⁰ is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while sulfides include —SR⁸⁰ groups, sulfoxides include —S(O)R⁸¹ groups, sulfones include —SO₂R⁸² groups, and sulfonyls include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “trifluoromethyldiazirido” refers to

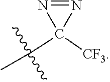 [SMILES: *C1(C(F)(F)F)N=N1]

The term “isocyano” refers to —NC.

The term “isothiocyano” refers to —NCS.

The term “pentafluorosulfanyl” refers to —SF5.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

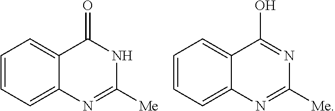

As another example, guanidines may exhibit the following isomeric forms in protic organic solution, also referred to as tautomers of each other:

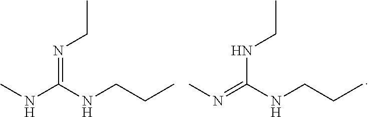

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Throughout this disclosure, various publications, patents and published patent specifications are referenced by an identifying citation. Also within this disclosure are Arabic numerals referring to referenced citations, the full bibliographic details of which are provided in sections within the Examples. The disclosures of these publications, patents and published patent specifications are hereby incorporated by reference into the present disclosure to more fully describe the present technology.

### The Present Technology

Radioligands targeting prostate-specific membrane antigen (PSMA) show encouraging efficacy in clinical imaging and therapy of prostate cancer. The first ligands to undergo clinical evaluation were antibodies targeting PSMA, but recently radiolabeled small molecule inhibitors of PSMA have gained traction due to their rapid accumulation in tumors, clearance from non-target tissue, and a side-effect profile deemed quite acceptable to patients. Many of these compounds employ a glutamate-urea-lysine or a glutamate-urea-glutamate moiety to achieve targeting of, and high affinity binding to PSMA. Among some of the PSMA-targeted ligands currently under clinical investigation are certain diagnostic compounds labeled with fluorine-18 or gallium-68 for tumor imaging by positron emission tomography (PET), or therapeutic compounds labeled with iodine-131, lutetium-177, bismuth-213, or actinium-225 for use in targeted radioligand therapy of PSMA-expressing cancers.

Although the tissue distribution and clearance of small molecules is typically rapid, the longer half-life of copper-64 (t_{1/2}=12.7 h) offers pharmacokinetic and logistical advantages over gallium-68 (t_{1/2}=68 min) or fluorine-18 (t_{1/2}=109 min), including the possibility for distribution of the radioligand from centralized production facilities. The longer half-life permits extended imaging, which might enhance detection of small metastatic lesions in areas with relatively high background. In spite of the lower probability of decay by positron emission of copper-64 (17.9%) compared to that of other radiometals such as gallium-68 (89%), the low energy of the emitted β⁺ enables high resolution PET imaging. Furthermore, copper-64 also decays by β⁻ emission (39.0%), facilitating a possible application to radioligand therapy. In this regard, copper-64 is itself a theranostic radioisotope.

The concept of theranostics describes the use of a matched pair of radionuclides to enable quantification of the distribution of radioactivity in the body, followed by radioligand therapy using the same delivery vector. In clinical targeted radioligand therapy using ¹⁷⁷Lu-labeled ligands targeting PSMA, indium-111 is usually used as a matched pair for lutetium-177 for preliminary dosimetry studies. Such a strategy is possible because on such compound, the DOTA macrocycle is able to stably chelate numerous trivalent radiometals, meaning that the chemical structure of the delivery vector, in principle, should remain largely the same. However, the affinity of the ligand for its target protein can change with a change of the metal to be complexed, which can thereby alter its tissue distribution, and potentially complicating estimates of dosimetry and pharmacokinetic models.

Copper-64 (t_{1/2}=12.7 h, β⁺=17.9%, β⁻=39.0%) and copper-67 (t_{1/2}=2.58 d, β⁻=100%) form an attractive theranostic pair. Copper-67 is a promising isotope for targeted radioligand therapy based on the close match between its physical half-life and its biological half-life when conjugated to many delivery vectors, its decay to a non-toxic daughter, and the tissue range of its emitted β⁻ particle, which is on the order of a few cell diameters in tissue. ⁶⁷Cu-Labeled radioligands show comparable efficacy to ¹⁷⁷Lu-labeled radioligands targeting the same receptor in preclinical models. Emerging approaches to the production of appropriate activities of copper-67 with high specific activity have revived interest in this theranostic radionuclide. The matched pair of copper-64 and copper-67 has been suggested to be advantageous to prospective dosimetry in targeted radioligand therapy, with a radiation safety profile comparable to currently used theranostic pairs. The attractive properties of Cu-64/Cu-67 have stimulated the application of ⁶⁴Cu-labeled PSMA-617 to prostate cancer imaging. Despite good targeting to metastatic lesions, uptake of radioactivity in the liver following administration of [⁶⁴Cu]Cu-PSMA-617 indicates sub-optimal in vivo stability that limits potential application of such DOTA-conjugated ligands. There remains a need in the art for better compounds that target cancer antigens and provide for theragnostics in the treatment and diagnosis of cancer. There is also a need for radiotherapeutic compounds that accumulate to a greater degree in tumors without unacceptable uptake in normal organs, as absorbed dose is a function of the integral of cumulative activity.

The present technology provides new compounds that overcome these problems, particularly providing trifunctional compounds having multiple domains, including one domain that targets and binds with relatively high affinity to tumor markers, and another domain that binds blood proteins such as serum albumin with a range of moderate to weak affinities. These compounds include a chelating moiety with a high selectivity for copper—specifically, derivatives of 3,6,10,13,16,19-hexaazabicyclo[6.6.6]icosane, known as sarcophagine.

Thus, in an aspect, a compound is provided that includes a tumor targeting domain (where the tumor targeting domain includes a moiety capable of recognizing or interacting with a molecular target on the surface of tumor cells), a blood-protein binding domain, and a sarcophagine-containing domain, where the moiety of the tumor targeting domain is distal to and sterically unimpeded by the blood-protein binding domain.

As discussed above, the tumor targeting domain (“TTD”) includes a moiety capable of recognizing or interacting with a molecular target on the surface of tumor cells. Such molecular targets include cell surface proteins such as receptors, enzymes, and antigens. For example, the molecular target may be a receptor, an enzyme, and/or an antigen expressed on a tumor cell surface (such as a tumor-specific cell surface protein) capable of interacting with the tumor targeting domain. An example of such a tumor targeting domain is the glutamate-urea-lysine motif recognized by prostate specific membrane antigen (PSMA) which is expressed on the surface of most prostate cancer cells. Another example is edotreotide, recognized by somatostatin receptors expressed on the surface of many neuroendocrine cancers. Thus, the tumor targeting domain of any aspect and embodiment herein may be capable of binding to a tumor associated molecular target that includes one or more of: a tumor associated molecular target that is a tumor-specific cell surface protein or other marker such as prostate specific membrane antigen (PSMA), somatostatin peptide receptor-2 (SSTR2), alphavbeta3 (αvβ3), alphavbeta6, a gastrin-releasing peptide receptor, a seprase (e.g., fibroblast activation protein alpha (FAP-alpha)), an incretin receptor, a glucose-dependent insulinotropic polypeptide receptor, VIP-1, NPY, a folate receptor, LHRH, a neuronal transporter (e.g., noradrenaline transporter (NET)), EGFR, HER-2, VGFR, MUC-1, CEA, MUC-4, ED2, TF-antigen, an endothelial specific marker, neuropeptide Y, uPAR, TAG-72, a claudin, a CCK analog, VIP, bombesin, VEGFR, a tumor-specific cell surface protein, GLP-1, CXCR4, Hepsin, TMPRSS2, a caspace, cMET, or an overexpressed peptide receptor. The preceeding are simply representative tumor associated molecular targets and for which detailed structural information exists for both the target and compounds that bind it. The various antibodies, peptides and compounds that display specific affinity for these particular cellular targets are widely described in the scientific literature, and can be adapted to the present technology as tumor targeting domains. The tumor targeting domain of any aspect and embodiment herein is capable of binding to the tumor associated molecular target with at least moderate affinity to high affinity (e.g., the equilibrium binding constant (K_{D}) ranging from about 10\hat{ }8 M to about 10\hat{ }10 M).

Thus, an example of a tumor targeting domain includes a moiety that targets and binds to the active site of PSMA, include for example, a glutamate-ureido-amino acid sequence, a glutamate-urea-lysine sequence with or without an aromatic substituent at the epsilon amine of lysine, or any derivative thereof that can bind the active site of PSMA with moderate to high affinity. Exemplary structures are provided herein, however other regions of PSMA can be targeted, and these are interchangeable with the PSMA tumor targeting domains in the compounds detailed herein. One exemplary copper-containing trifunctional compound that has affinity for PSMA is the following:

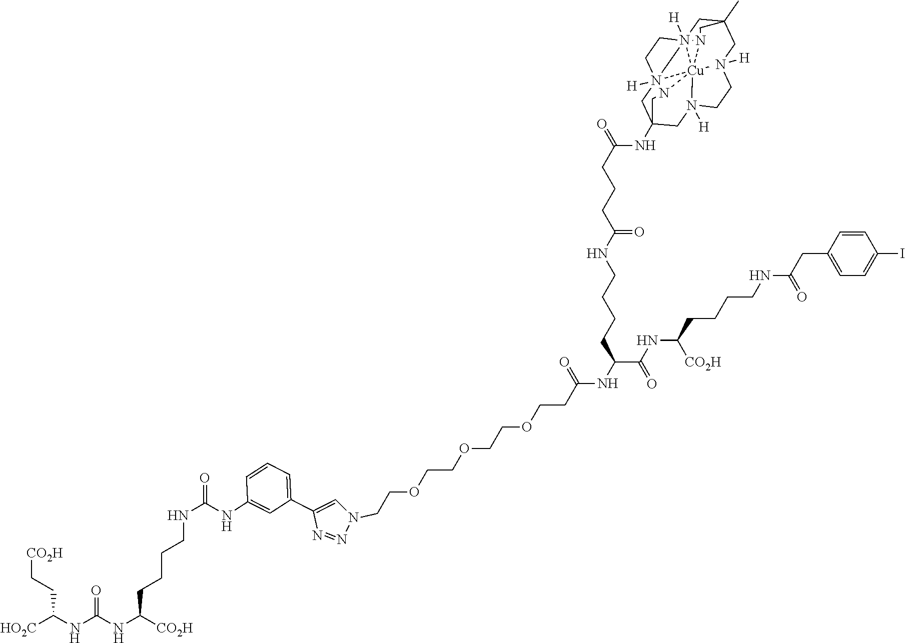

where the “Cu” may be ⁶⁴Cu⁺² or ⁶⁷Cu⁺².

Seprase (or Fibroblast Activation Protein (FAP)) is an integral membrane serine peptidase. In addition to gelatinase activity, seprase has a dual function in tumour progression. Seprase promotes cell invasiveness towards the ECM and also supports tumor growth and proliferation. As discussed above, the tumor targeting domain may include a moiety that binds to seprase, such as a seprase inhibitor. An exemplary structure of a copper-containing trifunctional compound that has affinity for FAP, and is useful in the diagnosis and therapy of most cancers, is provided below:

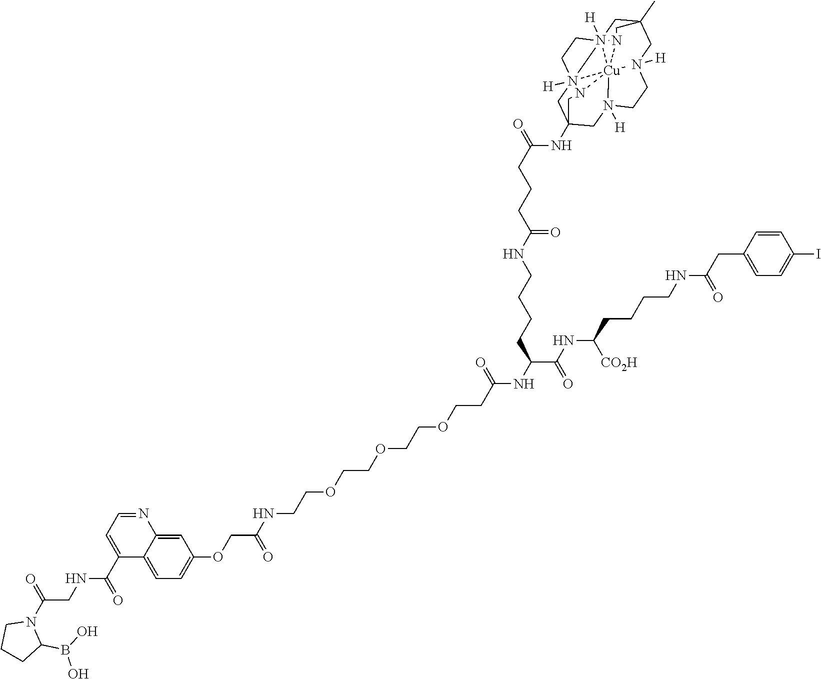

where the “Cu” may be ⁶⁴Cu⁺² or ⁶⁷Cu⁺².

Somatostatin is a peptide hormone that regulates the endocrine system and affects neurotransmission and cell proliferation via interaction with G protein-coupled somatostatin receptors and inhibition of the release of numerous secondary hormones. Somatostatin has two active forms produced by alternative cleavage of a single preproprotein. There are five known somatostatin receptors, all being G protein-coupled seven transmembrane receptors: SST1 (SSTR1); SST2 (SSTR2); SST3 (SSTR3); SST4 (SSTR4); and SST5 (SSTR5). Exemplary somatostatin receptor agonists include somatostatin itself, lanreotide, octreotate, octreotide, pasireotide, and vapreotide. Many neuroendocrine tumors express SSTR2 and the other somatostatin receptors. Long acting somatostatin agonists (e.g., Octreotide, Lanreotide) are used to stimulate the SSTR2 receptors, and thus to inhibit further tumor proliferation. See, Zatelli M C, et al., (Apr 2007). “Control of pituitary adenoma cell proliferation by somatostatin analogs, dopamine agonists and novel chimeric compounds”. European Journal of Endocrinology/European Federation of Endocrine Societies. 156 Suppl 1: S29-35. Octreotide is an octapeptide that mimics natural somatostatin but has a significantly longer half-life in vivo. Octreotide is used for the treatment of growth hormone producing tumors (acromegaly and gigantism), when surgery is contraindicated, pituitary tumors that secrete thyroid stimulating hormone (thyrotropinoma), diarrhea and flushing episodes associated with carcinoid syndrome, and diarrhea in people with vasoactive intestinal peptide-secreting tumors (VIPomas). Lanreotide is used in the management of acromegaly and symptoms caused by neuroendocrine tumors, most notably carcinoid syndrome. Pasireotide is a somatostatin analog with an increased affinity to SSTR5 compared to other somatostatin agonists and is approved for treatment of Cushing's disease and acromegaly. Vapreotide is is used in the treatment of esophageal variceal bleeding in patients with cirrhotic liver disease and AIDS-related diarrhea. Thus, with respect to the present technology, below is depicted an exemplary structure of a copper-containing trifunctional compound based on a derivative of lanreotide, that has affinity for SSTR2, and may be used in the diagnosis and therapy of the above-described maladies:

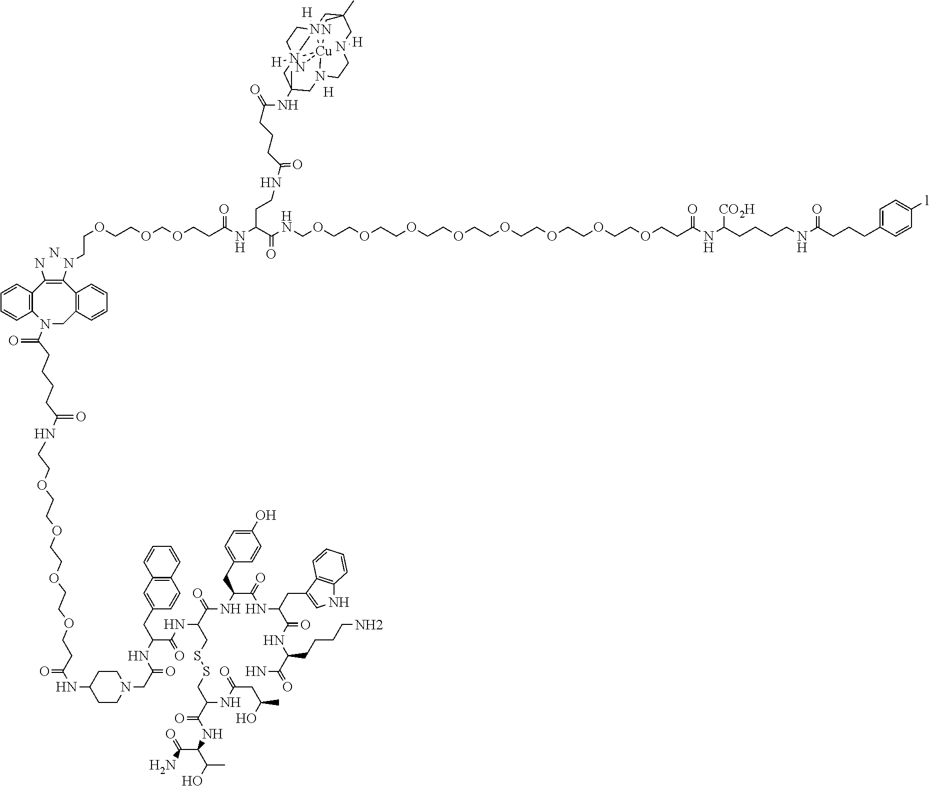

where the “Cu” may be ⁶⁴Cu⁺² or ⁶⁷Cu⁺².

Bombesin is a peptide originally isolated from the skin of the European fire-bellied toad (Bombina bombina). In addition to stimulating gastrin release from G cells, bombesin activates at least three different G-protein-coupled receptors: BBR1, BBR2, and BBR3, where such activity includes agonism of such receptors in the brain. Bombesin is also a tumor marker for small cell carcinoma of lung, gastric cancer, gallbladder, pancreatic cancer, and neuroblastoma. Bombesin receptor agonists include, but are not limited to, BBR-1 agonists, BBR-2 agonists, and BBR-3 agonists. An exemplary structure of a copper-containing trifunctional compound, that has affinity for bombesin and can be used in the diagnosis and therapy of the above cancers, is provided below:

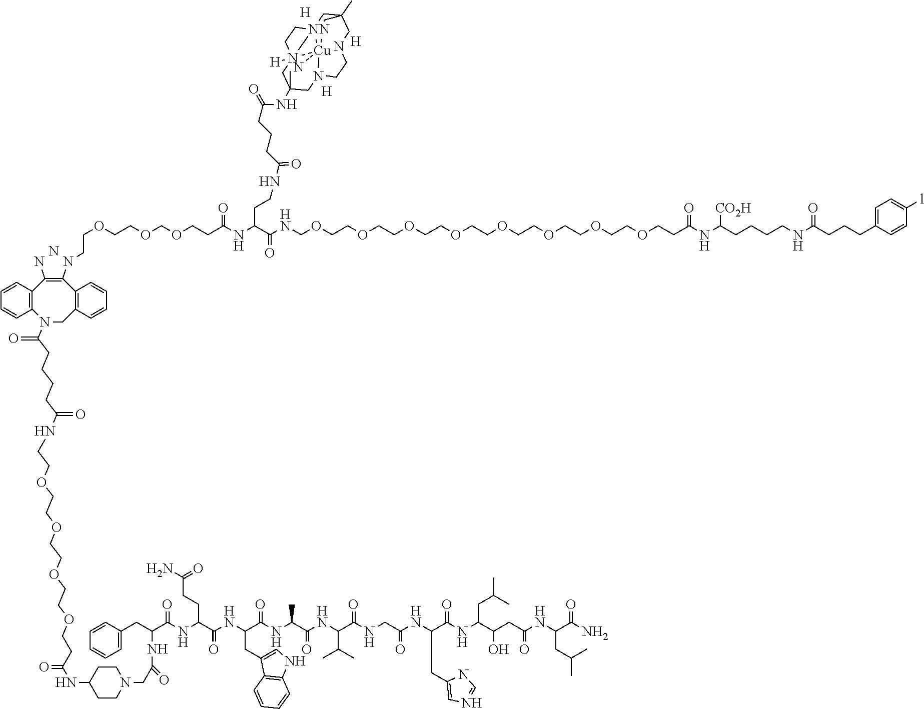

where the “Cu” may be 64Cu⁺² or 67Cu⁺².

Thus, the tumor targeting domain of any embodiment disclosed herein may include a modified antibody, modified antibody fragment, a modified binding peptide, a prostate specific membrane antigen (“PSMA”) binding peptide, a somatostatin receptor agonist, a bombesin receptor agonist, a seprase binding compound, or binding fragment of any one or more thereof. The tumor targeting domain of any embodiment disclosed herein may include belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab. The tumor targeting domain of any embodiment disclosed herein may include an antigen-binding fragment of belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab.

The blood-protein binding domain “BBD” (e.g., the albumin-binding domain; the albumin-binding moiety) plays a role in modulating the rate of blood plasma clearance of the compounds in a subject, thereby increasing circulation time and compartmentalizing the cytotoxic action of cytotoxin-containing domain and/or imaging capability of the imaging agent-containing domain in the plasma space instead of normal organs and tissues that may express antigen. Without being bound by theory, this component of the compound is believed to interact reversibly with serum proteins, such as albumin and/or cellular elements. The affinity of this blood-protein binding domain (e.g., the albumin-binding domain; the albumin-binding moiety) for plasma or cellular components of the blood may be configured to affect the residence time of the compounds in the blood pool of a subject. In any embodiment herein, the blood-protein binding domain (e.g., the albumin-binding domain; the albumin-binding moiety) may be configured so that it binds reversibly or non-reversibly with albumin when in blood plasma.

By way of example, the blood-protein binding domain of any aspect or embodiment herein may include a short-chain fatty acid, medium-chain chain fatty acid, a long-chain fatty acid, myristic acid, a substituted or unsubstituted indole-2-carboxylic acid, a substituted or unsubstituted thioamide, a substituted or unsubstituted 4-oxo-4-(5,6,7,8-tetrahydronaphthalen-2-yl)butanoic acid, a substituted or unsubstituted naphthalene acylsulfonamide, a substituted or unsubstituted diphenylcyclohexanol phosphate ester, a substituted or unsubstituted 4-iodophenylalkanoic acid, a substituted or unsubstituted 3-(4-iodophenyl)propionic acid, a substituted or unsubstituted 2-(4-iodophenyl)acetic acid, or a substituted or unsubstituted 4-(4-iodophenyl)butanoic acid. In any embodiment herein, it may be that the blood-protein binding domain is

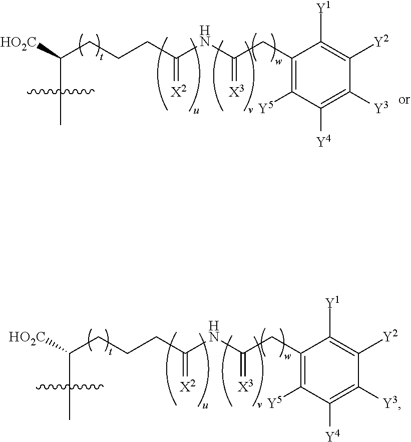

where Y¹, Y², Y³, Y⁴, and Y⁵ are independently at each occurrence H, halo, or alkyl; X² and X³ are each independently O or S, t is independently at each occurrence 0, 1, or 2; u is independently at each occurrence 0 or 1; v is independently at each occurrence 0 or 1; and w is independently at each occurrence 0, 1, 2, 3, or 4, optionally wherein u and v cannot be the same value. Certain representative examples of moieties that bind the blood protein albumin, that may be included in any embodiment herein, include one or more of the following:

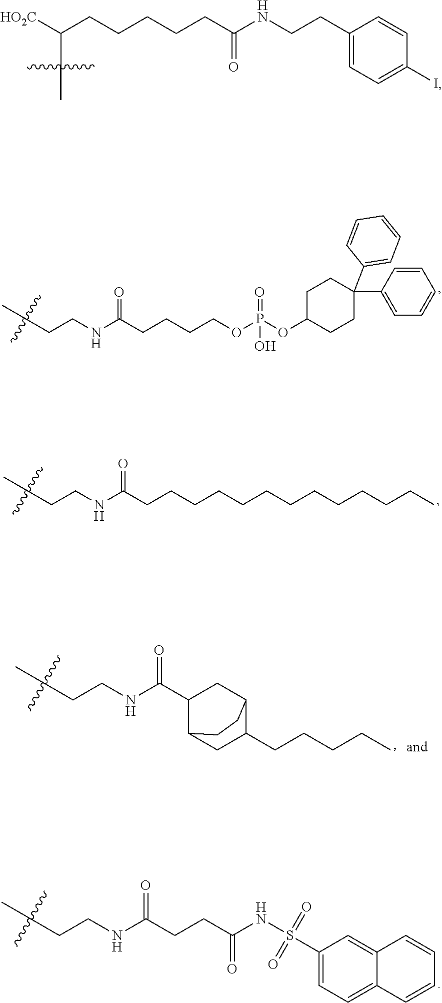 [SMILES: *C(CCCCCC(=O)NCCc1ccc(I)cc1)C(=O)O ; *CCNC(=O)CCCCOP(=O)(O)OC1CCC(c2ccccc2)(c2ccccc2)CC1 ; *CCNC(=O)CCCCCCCCCCCCC ; *CCNC(=O)C1CC2CCC1CC2CCCCC ; *CCNC(=O)CCC(=O)NS(=O)(=O)c1ccc2ccccc2c1]

In any embodiment of the present technology, the compound may be of any one of Formulas I-V:

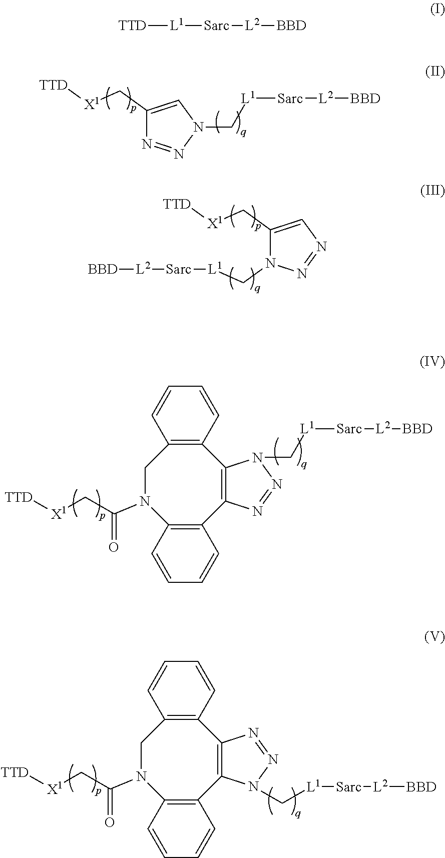

or a pharmaceutically acceptable salt and/or solvate thereof, where
TTD is the tumor targeting domain of any embodiment disclosed herein;
BBD is the blood-protein binding domain of any embodiment disclosed herein;
Sarc is the sarcophagine-containing domain of any embodiment disclosed herein;
X¹ is independently at each occurrence absent, O, S, NH, —C(O)—, —C(O)—NR¹—, —NR²—C(O)—, —C(O)—NR³—C₁-C₁₂ alkylene-, —C₁-C₁₂ alkylene-C(O)—, —C(O)—NR⁴—C₁-C₁₂ alkylene-C(O)—, -arylene-, -heterocyclene-, —O(CH₂CH₂O)ₐ—, —CH₂CH₂—O(CH₂CH₂O)_{b}—, —CH₂CH₂—O(CH₂CH₂O)_{c}—CH₂CH₂—, —O(CH₂CH₂O)_{d}—CH₂CH₂—, —C(O)—O(CH₂CH₂O)ₑ—, —O(CH₂CH₂O)_{f}—CH₂CH₂C(O)—, —C(O)—O(CH₂CH₂O)_{g}—, —C(O)—O(CH₂CH₂O)ₕ—CH₂CH₂—, —C(O)—O(CH₂CH₂O)ᵢ—CH₂CH₂C(O)—, —CH₂CH₂—O(CH₂CH₂O)ⱼ—CH₂CH₂C(O)—, —C(O)—NR—CH₂CH₂O(CH₂CH₂O)ₖ—, —C(O)—NR⁶—CH₂CH₂O(CH₂CH₂O)ₗ—CH₂CH₂—, —C(O)—NR—CH₂CH₂O(CH₂CH₂O)ₘ—CH₂CH₂C(O)—, an amino acid, a peptide of 2, 3, 4, 5, 6, 7, 8, 9, or 10 amino acids, or a combination of any two or more thereof, where a, b, c, d, e, f, g, h, i, j, k, l, and m are independently at each occurrence 0, 1, 2, 3, 4, 5, 6, 7, 8, 9, 10, 11, 12, 13, 14, 15, 16, 17, 18, or 19, and where R¹, R², R³, R⁴, R⁵, R⁶, and R⁷ are independently at each occurrence H, alkyl, or aryl;
L¹ is independently at each occurrence absent, O, S, NH, —C(O)—, —C(O)—NR⁸—, —NR⁹—C(O)—, —C(O)—NR¹⁰—C₁-C₁₂ alkylene-, —C₁-C₁₂ alkylene-C(O)—, —C(O)—NR¹¹—C₁-C₁₂ alkylene-C(O)—, -arylene-, -heterocyclene-, —O(CH₂CH₂O)_{a′}—, —CH₂CH₂—O(CH₂CH₂O)_{b′}—, —CH₂CH₂—O(CH₂CH₂O)_{c′}—CH₂CH₂—, —O(CH₂CH₂O)_{d′}—CH₂CH₂—, —C(O)—O(CH₂CH₂O)_{e′}—, —O(CH₂CH₂O)_{f′}—CH₂CH₂C(O)—, —C(O)—O(CH₂CH₂O)_{g′}—, —C(O)—O(CH₂CH₂O)_{h′}—CH₂CH₂—, —C(O)—O(CH₂CH₂O)_{i′}—CH₂CH₂C(O)—, —CH₂CH₂—O(CH₂CH₂O)_{j′}—CH₂CH₂C(O)—, —C(O)—NR¹²—CH₂CH₂O(CH₂CH₂O)_{k′}—, —C(O)—NR¹³—CH₂CH₂O(CH₂CH₂O)_{l′}—CH₂CH₂—, —C(O)—NR¹⁴—CH₂CH₂O(CH₂CH₂O)_{m′}—CH₂CH₂C(O)—, an amino acid, a peptide of 2, 3, 4, 5, 6, 7, 8, 9, or 10 amino acids, or a combination of any two or more thereof, where a′, b′, c′, d′, e′, f′, g′, h′, i′, j′, k′, l′, and m′ are independently at each occurrence 0, 1, 2, 3, 4, 5, 6, 7, 8, 9, 10, 11, 12, 13, 14, 15, 16, 17, 18, or 19, and where R⁸, R⁹, R¹⁰, R¹¹, R¹², R¹³, and R¹⁴ are independently at each occurrence H, alkyl, or aryl;
L² is independently at each occurrence absent, O, S, NH, —C(O)—, —C(O)—NR¹⁵—, —NR¹⁶—C(O)—, —C(O)—NR¹⁷—C₁-C₁₂ alkylene-, —C₁-C₁₂ alkylene-C(O)—, —C(O)—NR¹⁸—C₁-C₁₂ alkylene-C(O)—, -arylene-, -heterocyclene-, —O(CH₂CH₂O)_{a″}—, —CH₂CH₂—O(CH₂CH₂O)_{b″}—, —CH₂CH₂—O(CH₂CH₂O)_{c″}, —CH₂CH₂—, —O(CH₂CH₂O)_{d″}—CH₂CH₂—, —C(O)—O(CH₂CH₂O)_{e″}—, —O(CH₂CH₂O)_{f″}—CH₂CH₂C(O)—, —C(O)—O(CH₂CH₂O)_{g″}—, —C(O)—O(CH₂CH₂O)_{h″}—CH₂CH₂—, —C(O)—O(CH₂CH₂O)_{i″}—CH₂CH₂C(O)—, —CH₂CH₂—O(CH₂CH₂O)_{j″}—CH₂CH₂C(O)—, —C(O)—NR¹⁹—CH₂CH₂O(CH₂CH₂O)_{k″}—, —C(O)—NR²⁰—CH₂CH₂O(CH₂CH₂O)_{l″}, —CH₂CH₂—, —C(O)—NR²¹—CH₂CH₂O(CH₂CH₂O)_{m″}—CH₂CH₂C(O)—, an amino acid, a peptide of 2, 3, 4, 5, 6, 7, 8, 9, or 10 amino acids, or a combination of any two or more thereof, where a″, b″, c″, d″, e″, f″, g″, h″, i″, j″, k″, l″, and m″ are independently at each occurrence 0, 1, 2, 3, 4, 5, 6, 7, 8, 9, 10, 11, 12, 13, 14, 15, 16, 17, 18, or 19, and where R¹⁵, R¹⁶, R¹⁷, R¹⁸, R¹⁹, R²⁰, and R²¹ are independently at each occurrence H, alkyl, or aryl;
p is independently at each occurrence 0, 1, 2, 3, 4, or 5; and
q is independently at each occurrence 1 or 2.

In any embodiment of the present technology, the tumor targeting domain may be

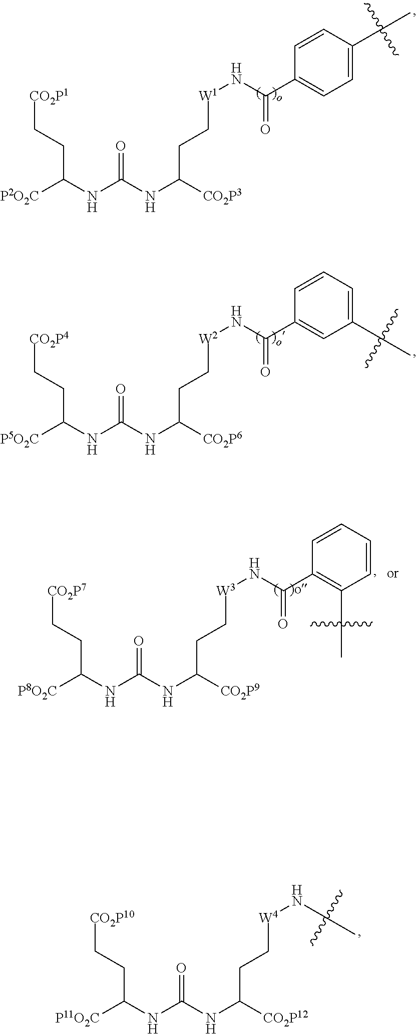

where W¹, W², W³, and W⁴ are each independently —C(O)—, —(CH₂)ᵣ—, or —(CH₂)ₛ—NH—C(O)—; r is independently at each occurrence 1 or 2; s is independently at each occurrence 1 or 2; P¹, P², P³, P⁴, P⁵, P⁶, P⁷, P⁸, P⁹, P¹⁰, P¹¹, and P¹² are each independently H, methyl, benzyl, 4-methoxybenzyl, or tert-butyl; and o, o′, and o″ are each independently 0 or 1. In any embodiment herein, it may be that P¹, P², P³, P⁴, P⁵, P⁶, P⁷, P⁸, P⁹, P¹⁰, P¹¹, and P¹² are each independently H or tert-butyl. In any embodiment herein, it may be that P¹, P², P³, P⁴, P⁵, P⁶, P⁷, P⁸, P⁹, P¹⁰, P¹¹, and P¹² are each independently H.

The sarcophagine-containing domain of the compounds of the present technology is the domain capable of chelating ⁶⁴Cu⁺² or ⁶⁷Cu⁺². In any embodiment disclosed herein, the sarcophagine-containing domain may be

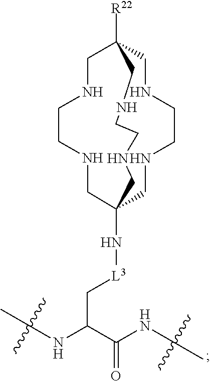

where R²² is H, alkyl, aryl, or NR²³R²⁴; R²³ and R²⁴ are each independently H, alkyl, aryl, alkanoyl, or aryloyl; L³ is absent, —C(O)—, —C₁-C₁₂ alkylene-, —C₁-C₁₂ alkylene-C(O)—, —NR²⁵C(O)—C₁-C₁₂ alkylene-C(O)—, —C₁-C₁₂ alkylene-NR²⁵C(O)—C₁-C₁₂ alkylene-C(O)—, -arylene-, —C₁-C₁₂ alkylene-C(O)NR²⁵—CH₂-phenylene-CH₂—, —C₁-C₁₂ alkylene-C(O)NR²⁵—CH₂-phenylene-C(O)—, —C₁-C₁₂ alkylene-NR²⁵C(O)—C₁-C₁₂ alkylene-C(O)—C(O)NR²⁵—CH₂-phenylene-CH₂—, or —C₁-C₁₂ alkylene-NR²⁵C(O)—C₁-C₁₂ alkylene-C(O)—C(O)NR²⁵—CH₂-phenylene-C(O)—; and R²⁵ is independently at each occurrence H, alkyl, or aryl. In any embodiment herein, it may be that R²² is H, methyl, or NH₂. In any embodiment herein, the sarcophagine-containing domain of the compound may chelate ⁶⁴Cu⁺² or ⁶⁷Cu⁺².

The present technology also provides compositions and medicaments comprising any one of the aspects and embodiments of the compounds of the present technology and a pharmaceutically acceptable carrier or one or more excipients or fillers (collectively referred to as “pharmaceutically acceptable carrier” unless otherwise specified). The compositions may be used in the methods and treatments described herein. The present technology also provides pharmaceutical compositions including a pharmaceutically acceptable carrier and an effective amount of a compound of any one of the aspects and embodiments of the compounds of the present technology for imaging and/or treating a condition; and where the condition may include one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer. For example, such conditions may include a mammalian tissue overexpressing PSMA, such as a cancer expressing PSMA (including cancer tissues, cancer related neo-vasculature, or a combination thereof), Crohn's disease, or IBD.

In a further related aspect, an imaging method is provided that includes administering a compound of any one of the aspects and embodiments of the compounds of the present technology (e.g., such as administering an effective amount) or administering a pharmaceutical composition comprising an effective amount of a compound of any one of the aspects and embodiments of the compounds of the present technology to a subject and, subsequent to the administering, detecting positron emission, detecting gamma rays from positron emission and annihilation (such as by positron emission tomography), and/or detecting Cerenkov radiation due to positron emission (such as by Cerenkov luminescene imaging). In any embodiment of the imaging method, the subject may be suspected of suffering from a condition that includes one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, a metastatic cancer, a mammalian tissue overexpressing PSMA, such as a cancer expressing PSMA (including cancer tissues, cancer related neo-vasculature, or a combination thereof), Crohn's disease, or IBD. The detecting step may occur during a surgical procedure on a subject, e.g., to remove a mammalian tissue overexpressing PSMA. The detecting step may include use of a handheld device to perform the detecting step. For example, Cerenkov luminescene images may be acquired by detecting the Cerenkov light using ultra-high-sensitivity optical cameras such as electron-multiplying charge-coupled device (EMCCD) cameras.

In any of the above embodiments, the effective amount may be determined in relation to a subject. “Effective amount” refers to the amount of a compound or composition required to produce a desired effect. One non-limiting example of an effective amount includes amounts or dosages that yield acceptable toxicity and bioavailability levels for therapeutic (pharmaceutical) use including, but not limited to, the treatment of e.g., one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer. Another example of an effective amount includes amounts or dosages that are capable of reducing symptoms associated with e.g., one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer, such as, for example, reduction in proliferation and/or metastasis. An effective amount of a compound of the present technology may include an amount sufficient to enable detection of binding of the compound to a target of interest including, but not limited to, one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer. Another example of an effective amount includes amounts or dosages that are capable of providing a detectable gamma ray emission from positron emission and annihilation (above background) in a subject with a tissue including one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, a metastatic cancer, and overexpressing PSMA, such as, for example, statistically significant emission above background. Another example of an effective amount includes amounts or dosages that are capable of providing a detectable Cerenkov radiation emission due to positron emission (above background) in a subject with a tissue including one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, a metastatic cancer, and overexpressing PSMA, such as, for example, statistically significant emission above background. The effective amount may be from about 0.01 μg to about 1 mg of the compound per gram of the composition, and preferably from about 0.1 μg to about 500 μg of the compound per gram of the composition.

As used herein, a “subject” or “patient” is a mammal, such as a cat, dog, rodent or primate. Typically the subject is a human, and, preferably, a human suffering from or suspected of suffering from one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer. The term “subject” and “patient” can be used interchangeably.

In particular, the effective amount of a compound of any embodiment herein for treating a cancer (such as one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer) and/or a mammalian tissue overexpressing PSMA may be from about 0.1 μg to about 50 μg per kilogram of the mass of the subject. Thus, for treating a cancer (e.g., one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer) and/or a mammalian tissue overexpressing PSMA; the effective amount of a compound of any embodiment described herein may be about 0.1 μg/kg, about 0.2 μg/kg, about 0.3 μg/kg, about 0.4 μg/kg, about 0.5 μg/kg, about 0.6 μg/kg, about 0.7 μg/kg, about 0.8 μg/kg, about 0.9 μg/kg, about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 22 μg/kg, about 24 μg/kg, about 26 μg/kg, about 28 μg/kg, about 30 μg/kg, about 32 μg/kg, about 34 μg/kg, about 36 μg/kg, about 38 μg/kg, about 40 μg/kg, about 42 μg/kg, about 44 μg/kg, about 46 μg/kg, about 48 μg/kg, about 50 μg/kg, or any range including and/or in between any two of these values.

In particular, the effective amount of a compound of any embodiment herein for imaging a cancer (such as one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer) and/or a mammalian tissue overexpressing PSMA may be from about 0.1 μg to about 50 μg per kilogram of the mass of the subject. Thus, for treating a cancer (e.g., one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer) and/or a mammalian tissue overexpressing PSMA; the effective amount of a compound of any embodiment described herein may be about 0.1 μg/kg, about 0.2 μg/kg, about 0.3 μg/kg, about 0.4 μg/kg, about 0.5 μg/kg, about 0.6 μg/kg, about 0.7 μg/kg, about 0.8 μg/kg, about 0.9 μg/kg, about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 22 μg/kg, about 24 μg/kg, about 26 μg/kg, about 28 μg/kg, about 30 μg/kg, about 32 μg/kg, about 34 μg/kg, about 36 μg/kg, about 38 μg/kg, about 40 μg/kg, about 42 μg/kg, about 44 μg/kg, about 46 μg/kg, about 48 μg/kg, about 50 μg/kg, or any range including and/or in between any two of these values.

The compounds of the present technology may also be administered to a patient along with other conventional imaging agents that may be useful in the imaging and/or treatment of one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, a metastatic cancer, or a mammalian tissue overexpressing PSMA. Such mammalian tissues include, but are not limited to, a cancer expressing PSMA (including cancer tissues, cancer related neo-vasculature, or a combination thereof), Crohn's disease, or IBD. Thus, a pharmaceutical composition and/or method of the present technology may further include an imaging agent different than the compounds of the present technology; a pharmaceutical composition and/or method of the present technology may include an treatment agent different than the compounds of the present technology; a pharmaceutical composition and/or method of the present technology may further include an imaging agent according to any embodiment of a compound of the present technology and therapeutic agent that is also according to any embodiment of a compound of the present technology. It may be that the compound according to the present technology is both a therapeutic agent and an imaging agent. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology may also include administering, either sequentially or in combination with one or more compounds of the present technology, a conventional imaging agent in an amount that can potentially or synergistically be effective for the imaging of one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, a metastatic cancer, and a mammalian tissue overexpressing PSMA.

In any of the embodiments of the present technology described herein, the pharmaceutical composition may be packaged in unit dosage form. The unit dosage form is effective in treating one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer. Generally, a unit dosage including a compound of the present technology will vary depending on patient considerations. Such considerations include, for example, age, protocol, condition, sex, extent of disease, contraindications, concomitant therapies and the like.

An exemplary unit dosage based on these considerations may also be adjusted or modified by a physician skilled in the art. For example, a unit dosage for a patient comprising a compound of the present technology may vary from 1×10⁻⁴ g/kg to 1 g/kg, preferably, 1×10⁻³ g/kg to 1.0 g/kg. Dosage of a compound of the present technology may also vary from 0.01 mg/kg to 100 mg/kg or, preferably, from 0.1 mg/kg to 10 mg/kg. Suitable unit dosage forms, include, but are not limited to powders, tablets, pills, capsules, lozenges. suppositories. patches. nasal sprays, injectibles, implantable sustained-release formulations, rnucoadherent films, topical varnishes, lipid complexes, etc.

The pharmaceutical compositions may be prepared by mixing one or more compounds of the present technology with pharmaceutically acceptable carriers, excipients, binders, diluents or the like to prevent and treat disorders associated with cancer (e.g., one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer). The compounds and compositions described herein may be used to prepare formulations and medicaments that treat e.g., one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer (such as castration resistant prostate cancer), a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer. Such compositions may be in the form of, for example, granules, powders, tablets, capsules, syrup, suppositories, injections, emulsions, elixirs, suspensions or solutions. The instant compositions may be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. The instant compositions may also include, for example, micelles or liposomes, or some other encapsulated form.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Various assays and model systems can be readily employed to determine the therapeutic effectiveness of the treatment according to the present technology.

For the indicated condition, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The examples herein are provided to illustrate advantages of the present technology and to further assist a person of ordinary skill in the art with preparing or using the compounds of the present technology or salts, pharmaceutical compositions, derivatives, prodrugs, or tautomeric forms thereof. The examples herein are also presented in order to more fully illustrate the preferred aspects of the present technology. The examples should in no way be construed as limiting the scope of the present technology, as defined by the appended claims.

The examples can include or incorporate any of the variations, aspects or embodiments of the present technology described above. The variations, aspects or embodiments described above may also further each include or incorporate the variations of any or all other variations, aspects or embodiments of the present technology.

### EXAMPLES

Materials and Instrumentation. All solvents and reagents, unless otherwise noted, were purchased from commercial sources and used as received without further purification. Solvents noted as “dry” were obtained following storage over 3 Åmolecular sieves. Reactions were monitored by thin-layer chromatography (TLC, Whatman UV254 aluminum-backed silica gel).

### Synthesis of RPS-085, [⁶⁴Cu]Cu-RPS-085 and [⁶⁷Cu]Cu-RPS-085

The trifunctional scaffold (((S)-5-(3-(3-(1-((14S,17S)-14-(4-aminobutyl)-17-carboxy-24-(4-iodophenyl)-12,15,23-trioxo-3,6,9-trioxa-13,16,22-triazatetracosyl)-1H-1,2,3-triazol-4-yl)phenyl)ureido)-1-carboxypentyl)carbamoyl)-L-glutamic acid was synthesized as described in Kelly et al., (Eur J Nucl Med Mol Imaging. 2018; 45: 1841-51). This amine (13 mg, 10 μmol, 1 eq) was dissolved in anhydrous DMF (1 mL; Sigma Aldrich, USA) and stirred for 5 min at room temperature (rt) with N,N-diisopropylethylamine (18 μL, 100 μmol, 10 eq).

Conjugation of the sarcophagine chelator to scaffold was achieved by a reaction between an NHS ester derivative of MeCOSar and the N^{ε} of Lys. An overview of this synthesis is provided below:

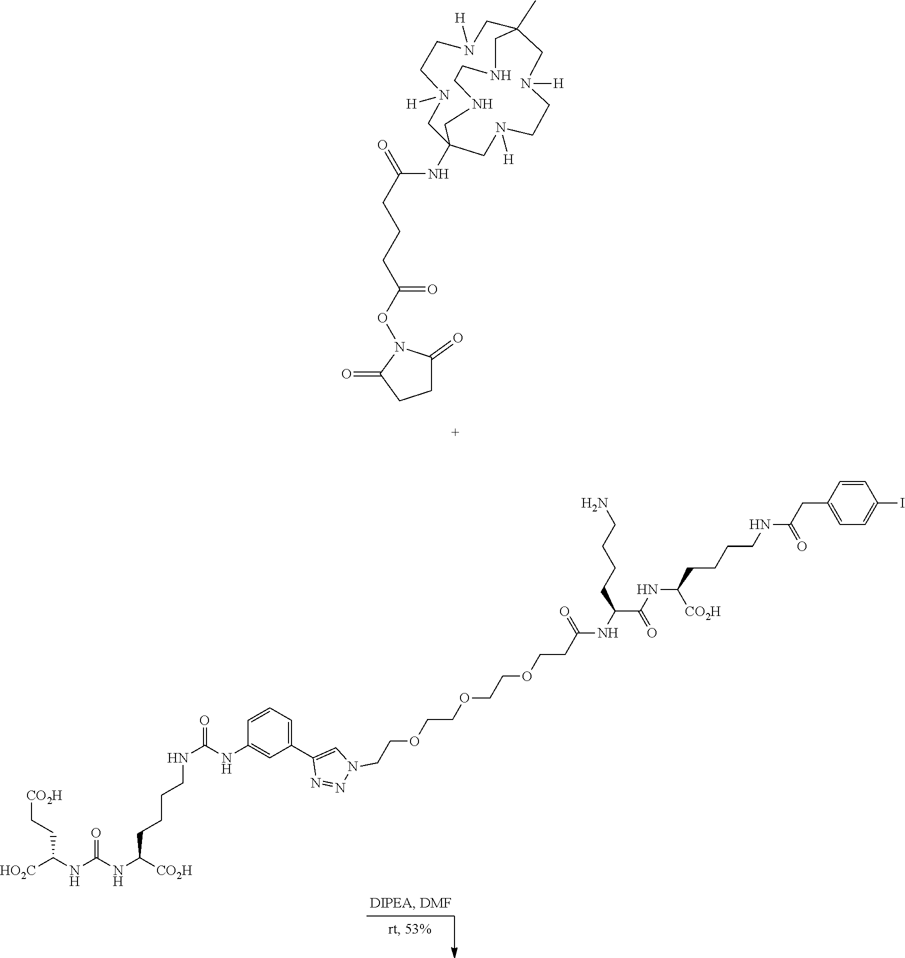 [SMILES: CC12CNCCNCC(NC(=O)CCCC(=O)ON3C(=O)CCC3=O)(CNCCNC1)CNCCNC2 ; NCCCC[C@H](NC(=O)CCOCCOCCOCCn1cc(-c2cccc(NC(=O)NCCCC[C@H](NC(=O)N[C@@H](CCC(=O)O)C(=O)O)C(=O)O)c2)nn1)C(=O)N[C@@H](CCCCNC(=O)Cc1ccc(I)cc1)C(=O)O]

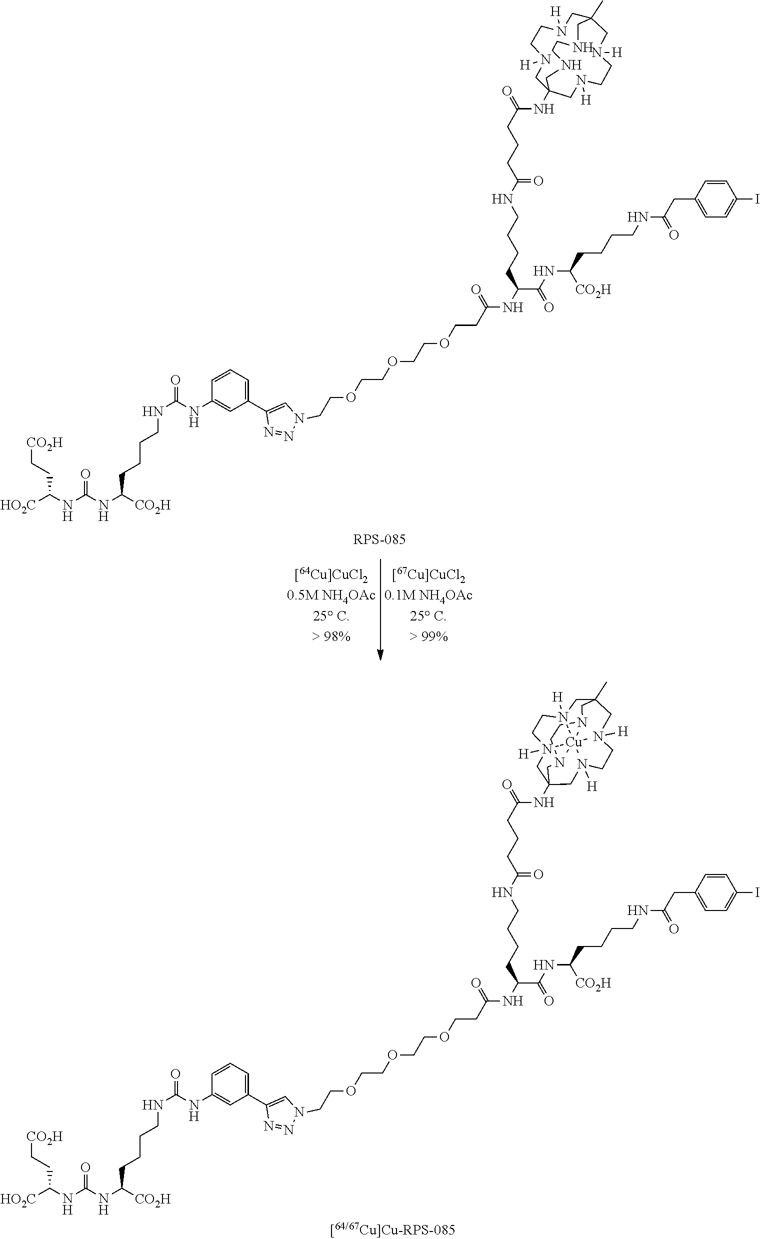

A solution of the N-succinimidyl ester of MeCOSar (6.2 mg, 12 μmol, 1.2 eq) in DMF (0.5 mL) was added dropwise to the reaction, and the resulting mixture was stirred for 5 hours at rt. The crude mixture was purified by HPLC using a dual pump Agilent 1200 Series HPLC equipped with a Phenomenex Luna® C18(2) 100 Å, 250 cm×21.2 mm I.D., 10 m reverse phase column. The mobile phase was a gradient of 10% acetonitrile (MeCN)/water (H₂O)+0.05% trifluoroacetic acid (TFA) to 90% MeCN/H₂O±0.05% TFA over 40 min at a flow rate of 12 mL/min. The peak corresponding to RPS-085 was collected and lyophilized. RPS-085 was isolated as a white powder (8.6 mg, 53%).

The purity of the product was confirmed by analytical HPLC using a dual pump Agilent ProStar HPLC fitted with an Agilent ProStar 325 Dual Wavelength UV-Vis Detector (Agilent Technologies, USA). UV absorption was monitored at 220 nm and 280 nm. Analysis was performed on an XSelect™ CSH™ C18 5 m 4.6×50 mm column (Waters, USA) using a gradient method at a flow rate of 2 mL/min. The gradient was: 0-1 min: 0% B; 1-8 min: 0-100% B; 8-9 min: 100% B; 9-10 min: 100-0% B. Mobile phase A consisted of H₂O±0.01% v/v TFA (Sigma Aldrich, USA), and mobile phase B consisted of 90% v/v MeCN/H₂O±0.01% TFA. The identity of the product was confirmed by mass spectrometry. Mass was determined using a Waters ACQUITY UPLC® coupled to a Waters SQ Detector 2 (Waters, USA). MS (ESI+): 1620.14. Calculated mass: 1618.74.

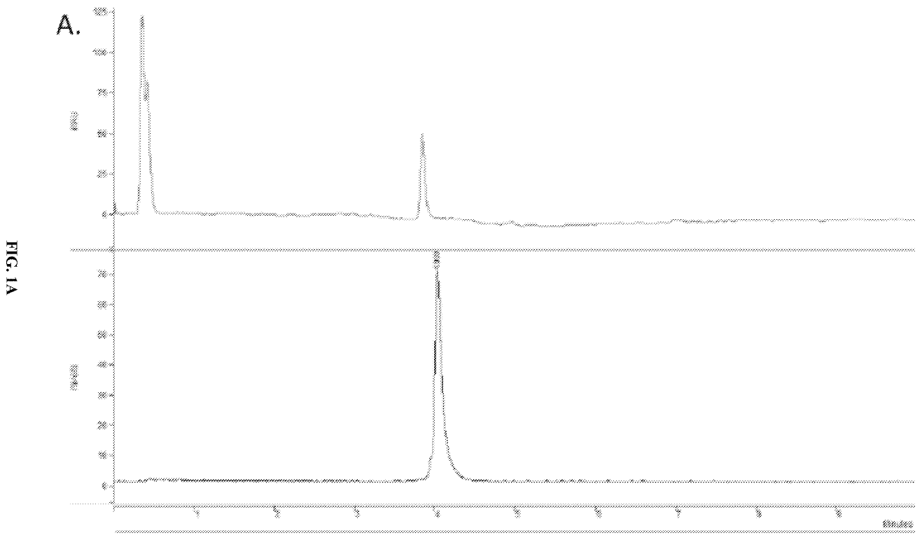

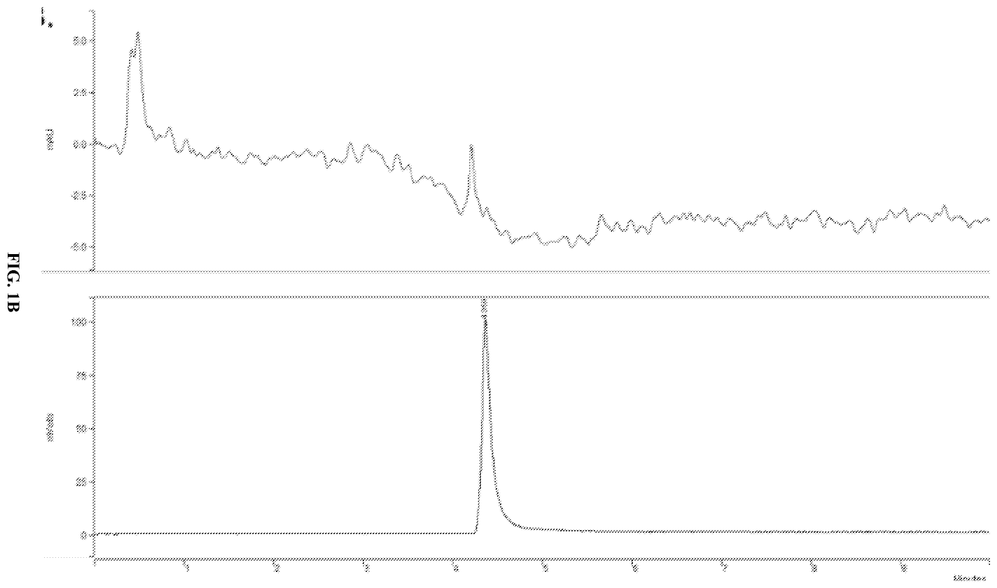

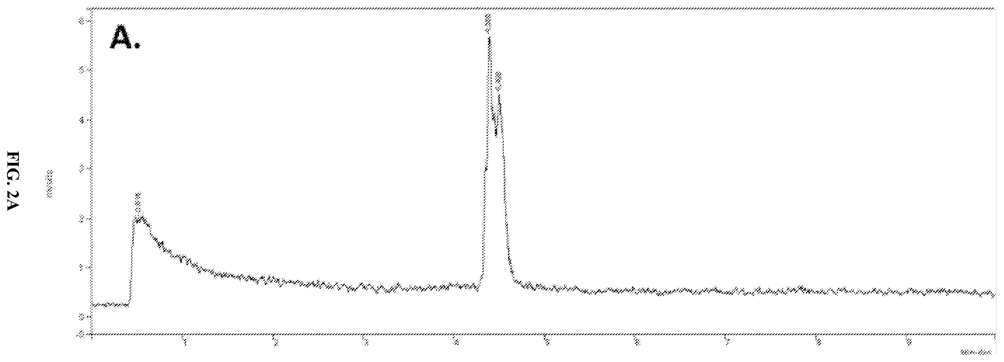

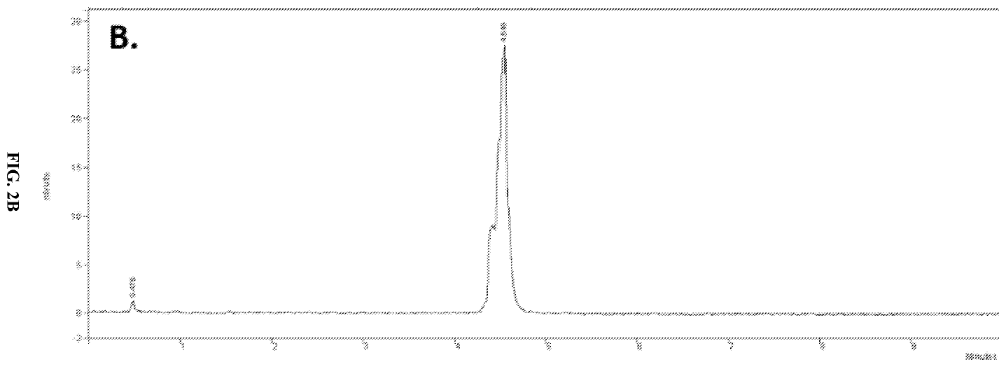

Relative to parent compound RPS-063, the resulting RPS-085 compound's affinity for PSMA was reduced by approximately one order of magnitude. Metal-free RPS-085 inhibited PSMA with an IC₅₀=29.1±2.4 nM. Affinity for human serum albumin (HSA) was also decreased, with K_{d}=9.9±1.7 μM.

[⁶⁴Cu]Cu-RPS-085 was quantitatively radiolabeled (n=6) in 20 min at 25° C. in both 10×PBS (pH 7.4) and 0.5 M NH₄OAc (pH 5.5). At the same temperature, labeling in 3 N NaOAc (pH 4.5) was accomplished in 35±2% yield (n=2). [⁶⁴Cu]CuCl₂ was purchased as a solution in dilute HCl from the University of Wisconsin. An aliquot of the [⁶⁴Cu]CuCl₂ solution (50-100 μL containing 350-800 MBq) was transferred to an Eppendorf tube, and diluted with 300 μL 0.5 M NH₄OAc. To this solution was added 5 μL of a 1 mg/mL solution of RPS-085 in DMSO. The reaction was incubated at 25° C. for 20 min in an Eppendorf ThermoMixer® C (VWR, USA). Then the sample was diluted to 10 mL with H₂O and passed through a pre-conditioned Sep-Pak C18 Plus Light cartridge (Waters, USA). The reaction vessel and cartridge were washed with 5 mL H₂O. The activity retained on the cartridge was eluted with 100 μL EtOH (300 proof, VWR, USA) followed by 900 μL saline (0.9% NaCl solution; VWR, USA). Radiochemical purity was determined by analytical reverse phase (radio)HPLC using a dual-pump Varian Dynamax HPLC system (Agilent Technologies, USA) fitted with a dual UV-Vis detector, and radiochemical purity was determined using a NaI(Tl) flow count detector (Bioscan, USA). UV absorption was monitored at 220 nm and 280 nm. Analyses were performed on a Symmetry C18 column (5 m, 4.6×50 mm, 100 Å; Waters, USA) using a gradient method at a flow rate of 2 mL/min. The gradient and mobile phase composition was the same as described above. The molar activity varied according to the activity of [⁶⁴Cu]Cu²⁺. When the starting activity was 800 MBq, [⁶⁴Cu]Cu-RPS-085 was isolated with a molar activity of 117 GBq/μmol and radiochemical purity >99% (FIGS. 1A-1B).

[⁶⁷Cu]Cu-RPS-085 was quantitatively radiolabeled at a concentration of 6.2 M in 0.1 M NH₄OAc in 20 min at 25° C. The pH of the reaction mixture was approximately 6. Cu-67 was supplied by the Isotope Program within the Office of Nuclear Physics in the Department of Energy's Office of Science. The activity was diluted with 100 μL 0.1 M NH₄OAc to achieve a radioactivity concentration of approximately 4 GBq/mL. A 60 μL aliquot of the solution (containing 245 MBq) was added to a solution of 10 μL of a 1 mg/mL solution of RPS-085 in DMSO diluted with 930 μL 0.1 M NH₄OAc. The reaction was incubated at 25° C. for 20 min in an Eppendorf ThermoMixer® C (VWR, USA). Then the sample was diluted to 10 mL with H₂O and passed through a pre-conditioned Sep-Pak C18 Plus Light cartridge (Waters, USA). The reaction vessel and cartridge were washed with 5 mL H₂O. The activity retained on the cartridge was eluted with 200 μL EtOH (300 proof, VWR, USA) followed by 1.8 mL saline (0.9% NaCl solution; VWR, USA). Radiochemical purity was determined by analytical reverse phase (radio)HPLC as described above. With a starting activity of 245 MBq, [⁶⁷Cu]Cu-RPS-085 was isolated with a molar activity of 41 GBq/μmol and a radiochemical purity >99% (FIGS. 2A-2B). By comparison, under the same labeling conditions, [⁶⁷Cu]Cu-RPS-063 was isolated with a radiochemical yield of 43% and a radiochemical purity >99%.

Stability Testing of the Compounds

The stability of the reformulated [⁶⁴Cu]Cu-RPS-085 solution was determined in triplicate at 25° C. Three 1 mL samples at a radioactivity concentration of approximately 10 MBq/mL and radiochemical purity >99% were transferred to Eppendorf tubes (VWR, USA). The samples were incubated for 24 h at 25° C. in an Eppendorf ThermoMixer® C (VWR, USA). Radiochemical purity was determined by analytical reverse phase (radio)HPLC and expressed as a fraction of the purity of the sample prior to incubation. After reformulation, [⁶⁴Cu]Cu-RPS-085 was stable at room temperature for more than 24 h.

The plasma stability of [⁶⁷Cu]Cu-RPS-085 was determined according to previously described methods (Alt, et al., Mol Pharmaceutics. 2014; 11: 2855-63). Briefly, frozen human plasma was purchased from Sigma Aldrich (USA), thawed at 37° C., and 200 μL aliquots were transferred to Eppendorf tubes. A 100 μL aliquot of [⁶⁷Cu]Cu-RPS-085 in 10% EtOH/saline was added to each tube, and the mixture was shaken at 300 rpm for 24 h at 37° C. on an Eppendorf ThermoMixer® C (VWR, USA). The experiment was performed in triplicate. The proteins were precipitated by addition of 600 μL acetonitrile. The samples were centrifuged at 13500 rpm for 3 min in an Eppendorf 5424-R Centrifuge. The supernatant was analyzed by analytical reverse phase (radio)HPLC as described above. After 24 h incubation in human plasma, [⁶⁷Cu]Cu-RPS-085 was 97.4±0.4% intact. The lone radiochemical impurity was an unidentified fragment of the parent compound. No uncomplexed [⁶⁷Cu]Cu²⁺ was observed.

Cell Culture, In Vitro Determination of IC₅₀ and Affinity for Human Serum Albumin

The PSMA-expressing human prostate cancer cell line, LNCaP, was obtained from the American Type Culture Collection. Cell culture supplies were obtained from Invitrogen (USA) unless otherwise noted. LNCaP cells were maintained in RPMI-1640 medium supplemented with 10% fetal bovine serum (Hyclone), 4 mM L-glutamine, 1 mM sodium pyruvate, 10 mM N-2-hydroxyethylpiperazine-N-2-ethanesulfonic acid (HEPES), 2.5 mg/mL D-glucose, and 50 g/mL gentamicin in a humidified incubator at 37° C./5% CO₂. Cells were removed from flasks for passage or for transfer to 12-well assay plates by incubating them with 0.25% trypsin/ethylenediaminetetraacetic acid (EDTA).

The IC₅₀ of metal-free RPS-085 was determined in a multi-concentration competitive binding assay against ^{99m}Tc-MIP-1427 (Hillier et al., J Nucl Med. 2013; 54: 1369-76) for binding to PSMA on LNCaP cells, according to previously described methods (Kelly et al., Eur J Nucl Med Mol Imaging. 2018; 45: 1841-51). RPS-085 was added in to the wells in final concentrations ranging from 1 pM-10 μM. The assay was performed in triplicate, and the IC₅₀ was expressed as mean±standard deviation.

Affinity for human serum albumin was determined by high-performance affinity chromatography, as previously described (Kelly et al., J Nucl Med. 2019; 60: 656-63). Briefly, [⁶⁷Cu]Cu-RPS-085 loaded onto a Chiralpak HSA analytic high-performance liquid chromatography column, 100×2 mm, 5 mm (Daicel Corp.), as a solution in 10% v/v EtOH/saline, with a maximum injected mass of 80 ng and a maximum injected volume of 20 μL. Analyses were performed in quadruplicate using an isocratic mobile phase of 5% v/v isopropanol/0.067 M phosphate buffer, pH 7.4 at a constant flow of 0.3 mL/min. The retention time of [⁶⁷Cu]Cu-RPS-085 was 4.64±0.52 min. K_{d} was determined using the equation: Kd=48.5×e^{-tR/2.85}+0.41, previously derived for the analytical conditions. The retention time of [⁶⁷Cu]Cu-RPS-085 was corrected for the offset between the UV detector and the radiation detector. The apparent K_{d} value was derived from a standard curve defined by the retention times of nonradioactive standards of known affinity for albumin.

LNCaP Xenograft Mouse Model and microPET/CT Imaging

All animal studies were approved by the Institutional Animal Care and Use Committee of Weill Cornell Medicine and were undertaken in accordance with the guidelines set forth by the USPHS Policy on Humane Care and Use of Laboratory Animals. The mice were housed under standard conditions in approved facilities with 12 h light/dark cycles. Food and water was provided ad libitum throughout the course of the studies. Male BALB/c athymic nu/nu mice were purchased from the Jackson Laboratory (USA). Prior to inoculation, LNCaP cells were suspended at a density of 4×10⁷ cells/mL in a 1:1 mixture of PBS (VWR, USA):Matrigel (BD Biosciences, USA). Each mouse was injected subcutaneously in the left flank with 0.25 mL of the cell suspension. The animals were monitored twice weekly until palpable tumors emerged.

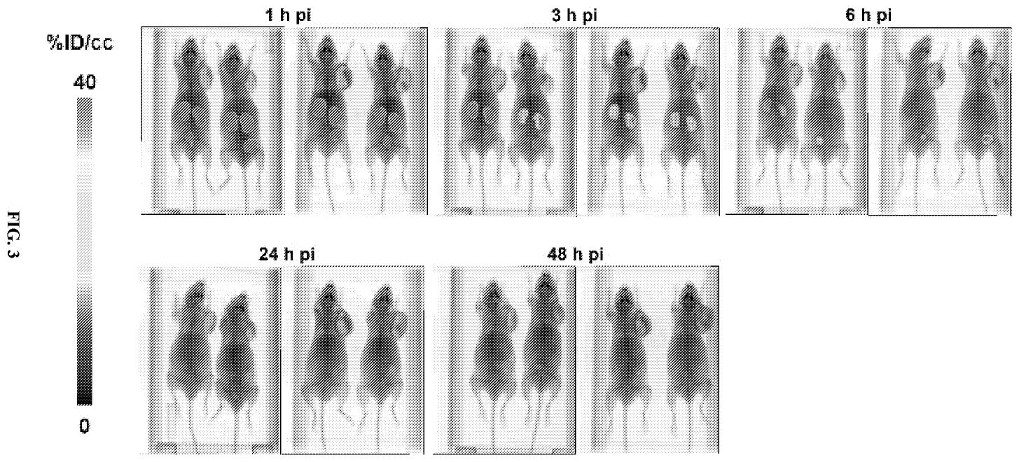

LNCaP xenograft tumors were clearly visualized with [⁶⁴Cu]Cu-RPS-085 (FIG. 3). Renal clearance was the primary route of excretion, leading to initial uptake of the compound in kidneys and bladder. Background tissue activity was low. Uptake in tumor and kidneys was estimated by drawing ROIs around each tissue. According to this semi-quantitative approach, activity in the tumor was greatest at 3 h p.i., but was virtually stable over the 48 h imaging window. By 6 h p.i., uptake in tumors exceeded kidneys, and by 24 h only tumors could be visualized by microPET/CT.

When tumor size was in the range 200-500 mm³, mice (n=4) were administered 21.2±0.5 MBq [⁶⁴Cu]Cu-RPS-085 by intravenous injection. The total mass dose received by each animal was approximately 300 ng (185 pmol). The mice were imaged under isoflurane anesthesia at 1 h, 3 h, 6 h, 24 h, and 48 h post injection (p.i.) by microPET/CT (Inveon™; Siemens Medical Solutions, USA). Total acquisition time was 30 min. A CT scan was obtained immediately before the PET acquisition for both anatomical co-registration and attenuation correction. Images were reconstructed using the Inveon™ software provided by the vendor.

Biodistribution and Dosimetry

When tumor size reached approximately 200 mm³, mice (n=4/time point) were administered 3.8±0.1 MBq [⁶⁴Cu]Cu-RPS-085 or 0.8±0.005 MBq [⁶⁷Cu]Cu-RPS-085 by intravenous injection. The total mass dose received by each animal was approximately 50 ng (31 pmol) and approximately 28 ng (17 pmol) for the [⁶⁴Cu]Cu-RPS-085 and [⁶⁷Cu]Cu-RPS-085 studies, respectively. The mice were sacrificed at 4 h, 24 h, or 48 h p.i. ([⁶⁴Cu]Cu-RPS-085) or 4 h, 24 h, and 96 h ([⁶⁷Cu]Cu-RPS-085). A blood sample was collected, and the following tissues were collected and weighed wet using a digital balance: heart, lungs, liver, stomach, small intestine, large intestine, spleen, pancreas, kidneys, muscle, bone, and tumor. The tissues were counted on a Wizard2 Automatic Gamma Counter (Perkin Elmer, USA) against a 1% injected dose standard. The counts were corrected for decay and for activity injected, and tissue uptake was expressed as percent injected dose per gram (% ID/g). Standard error of the mean (SEM) was calculated for each data point. Statistical analyses were performed using an unpaired t test with GraphPad Prism software. A P value of less than 0.05 was considered significant. The concentration of [67Cu]Cu-RPS-085 found in blood, heart, lungs, liver, small intestine, large intestine, stomach, spleen, pancreas, kidneys, muscle, and bone at 4 h, 24 h, and 96 h, post injection (n=4 per time point) is shown in Table 1 below. Published values for [177Lu]Lu-RPS-063 are shown for comparison; all concentrations are expressed as percent injected dose per organ.

TABLE 1
Dosimitry
Concentration of [⁶⁷Cu]Cu-RPS-085

| | 4 h | 24 h | 96 h |
|---|---|---|---|
| Blood | 0.020573 | 0.005816 | 0.003526 |
| Heart | 0.010779 | 0.0068 | 0.008094 |
| Lungs | 0.035801 | 0.018812 | 0.010634 |
| Liver | 0.279482 | 0.181086 | 0.120761 |
| Small Intestine | 0.149305 | 0.086516 | 0.060905 |
| Large Intestine | 0.289989 | 0.135395 | 0.137912 |
| Stomach | 0.055469 | 0.042278 | 0.044829 |
| Spleen | 0.027316 | 0.008292 | 0.005006 |
| Pancreas | 0.00535 | 0.002143 | 0.003044 |
| Kidneys | 11.63016 | 1.857067 | 0.685429 |
| Muscle | 0.002757 | 0.001018 | 0.000956 |
| Bone | 0.003853 | 0.001592 | 0.001075 |

Concentration of [⁶⁷Cu]Cu-RPS-063

| | 4 h | 24 h | 96 h |
|---|---|---|---|
| Blood | 0.365823 | 0.049244 | 0.012355 |
| Heart | 0.060967 | 0.022778 | 0.012206 |
| Lungs | 0.15797 | 0.04045 | 0.017779 |
| Liver | 0.473744 | 0.168577 | 0.095364 |
| Small Intestine | 0.280837 | 0.111912 | 0.05036 |
| Large Intestine | 0.852188 | 0.357323 | 0.20188 |
| Stomach | 0.116862 | 0.157667 | 0.080529 |
| Spleen | 0.297362 | 0.059259 | 0.024474 |
| Pancreas | 0.060834 | 0.023207 | 0.012 |
| Kidneys | 67.14676 | 17.98961 | 1.833882 |
| Muscle | 0.030731 | 0.017644 | 0.010552 |
| Bone | 0.023686 | 0.01768 | 0.010617 |

The data for the dosimetry calculation were based on an average of 4 to 5 animals at each time point at 4, 24 and 96 h post injection. First obtained was the percent injected dose per organ in blood, heart, lungs, liver, small intestine, large intestine, stomach, spleen, pancreas, kidneys, muscle, bone, tumor and tail. The biodistribution data was fitted using a power function over the first 96 h. The power function of each organ was used to interpolate the concentration at intervals of 2 h to give a better estimate of the kinetics. The integration time was extended for an additional 96 h with the assumption that the percent injected dose per organ was constant after the first 96 h and the only change in concentration between 96 h and 192 h was due to radioactive decay. A trapezoidal approximation was then used to obtain the integral over the two-hour intervals. The interval integrals were scaled to the value of the full integral to give a better estimate. These residence times were used to estimate the absorbed dose to a human subject using the OLINDA program with the adult human male model and no bladder clearance. The dose to the rest of the body was not used in this calculation.

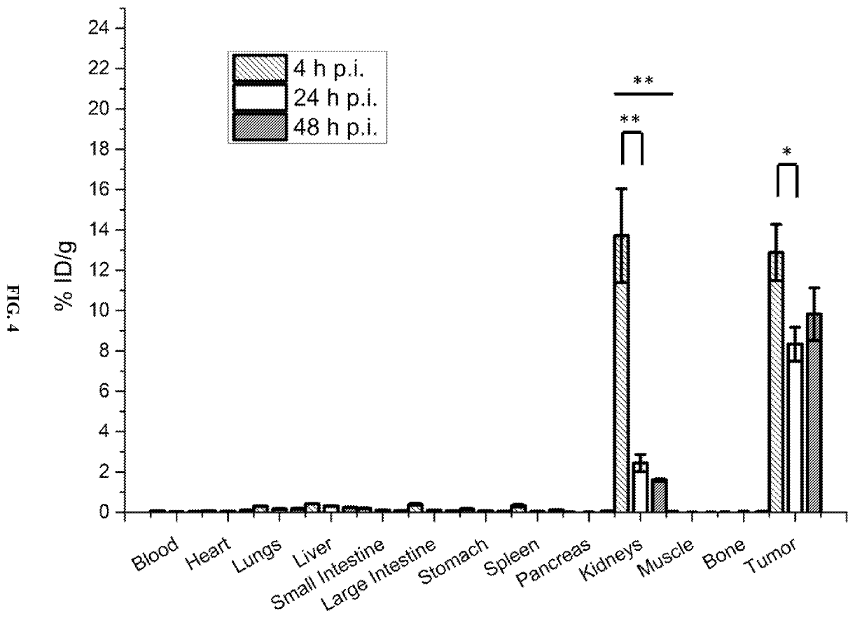

Uptake of [⁶⁴Cu]Cu-RPS-085 in tumors and tumor-to-background ratios were quantified following biodistribution studies. FIG. 4 shows the biodistribution of [⁶⁴Cu]Cu-RPS-085 in male Balb/C nu/nu mice bearing LNCaP xenografts. Mice (n=4/time point) were administered 3.8±0.1 MBq [⁶⁴Cu]Cu-RPS-085 intravenously and sacrificed at 4, 24, or 48 h p.i. Activity in each tissue was determined by comparison to a 1% ID activity standard and expressed as % ID/g±SEM. Statistically significant differences are indicated by * (P<0.05) and ** (P<0.01). Peak tumor uptake was observed at 4 h p.i. (12.9±1.4% ID/g), but clearance at 24 h (8.3±0.8% ID/g) and 48 h p.i. (9.8±1.3% ID/g) was not statistically significant (P>0.15). By comparison, activity in kidneys cleared from 13.7±2.3% ID/g at 4 h p.i. to 2.4±0.4% ID/g at 24 h p.i., with further clearance to 1.6±0.1% ID/g at 48 h p.i. Activity in all other tissues including liver was less than 0.5% ID/g at all time points. The distribution of [⁶⁴Cu]Cu-RPS-085 resulted in a tumor-to-kidney ratio of 0.9±0.2 at 4 h p.i., which significantly increased to 3.4±0.7 at 24 h p.i. and 6.1±0.8 at 48 h p.i. (P<0.02). Tumor-to-blood was 230±33 at 4 h p.i. and exceeded 400 subsequently. The tumor-to-muscle ratio was greater than 500 at all observed time points.

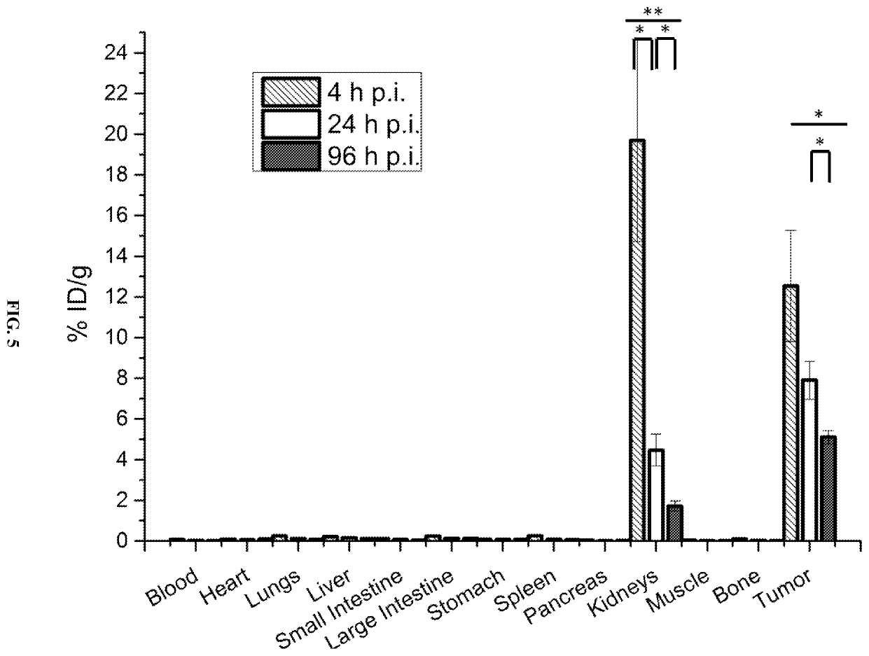

FIG. 5 shows the biodistribution of [⁶⁷Cu]Cu-RPS-085 in male Balb/C nu/nu mice bearing LNCaP xenografts. Mice (n=4/time point) were administered 0.8±0.005 MBq [⁶⁷Cu]Cu-RPS-085 intravenously and sacrificed at 4, 24, or 96 h p.i. Activity in each tissue was determined by comparison to a 1% ID activity standard and expressed as % ID/g±SEM. Statistically significant differences are indicated by * (P<0.05) and ** (P<0.01). The biodistribution of [⁶⁷Cu]Cu-RPS-085 closely mimics that of [⁶⁴Cu]Cu-RPS-085 at 4 h and 24 h p.i. The major tissues in which the radioligand accumulated were tumor and kidneys (FIG. 5). At these time points, tumor uptake was 12.5±2.7% ID/g and 7.9±0.9% ID/g, respectively. Activity in the kidney was 19.7±5.0% ID/g and 4.5±0.8% ID/g, respectively. These values were not significantly higher than those obtained in the [⁶⁴Cu]Cu-RPS-085 experiments (P>0.06). By 96 h p.i., activity in the tumor decreased to 5.1±3.3% ID/g, while activity in the kidney was 1.7±0.3% ID/g. The tumor-to-kidney ratio at this time point was 3.0±0.5. The estimated absorbed dose in tumors over 192 h is 14319 mSv/MBq. By comparison, the absorbed dose in kidneys is 884 mSv/MBq, representing a decrease of more than 16-fold. The whole body absorbed dose is 14.5 mSv/MBq, with no other tissue exceeding 65 mSv/MBq (Table 2).

TABLE 2
Whole body and organ-specific effective dose (mrem/mCi) and absorbed dose

| | | | | | Effective | Absorbed |
|---|---|---|---|---|---|---|
| | | | | | Dose | Dose |
| Target | | | | | (mrem/ | (mSv/ |
| Organ | Alpha | Beta | Photon | Total | mCi) | MBq) |
| Adrenals | 0.00E+00 | 0.00E+00 | 1.88E−02 | 1.88E−02 | 18.8 | 7.0 |
| Brain | 0.00E+00 | 0.00E+00 | 1.11E−05 | 1.11E−05 | 0.0111 | 0.0 |
| Breasts | 0.00E+00 | 0.00E+00 | 7.32E−04 | 7.32E−04 | 0.732 | 0.3 |
| Gallbladder | 0.00E+00 | 0.00E+00 | 1.23E−02 | 1.23E−02 | 12.3 | 4.6 |
| Wall | | | | | | |
| LLI Wall | 0.00E+00 | 1.57E−01 | 1.81E−02 | 1.75E−01 | 175 | 64.8 |
| Small | 0.00E+00 | 0.00E+00 | 8.97E−03 | 8.97E−03 | 8.97 | 3.3 |
| Intestine | | | | | | |
| Stomach | 0.00E+00 | 0.00E+00 | 7.30E−03 | 7.30E−03 | 7.3 | 2.7 |
| Wall | | | | | | |
| ULI Wall | 0.00E+00 | 5.01E−02 | 9.62E−03 | 5.97E−02 | 59.7 | 22.1 |
| Heart | 0.00E+00 | 9.24E−03 | 3.04E−03 | 1.23E−02 | 12.3 | 4.6 |
| Wall | | | | | | |
| Kidneys | 0.00E+00 | 2.26E+00 | 1.22E−01 | 2.39E+00 | 2390 | 884.3 |
| Liver | 0.00E+00 | 2.43E−02 | 1.07E−02 | 3.49E−02 | 34.9 | 12.9 |
| Lungs | 0.00E+00 | 4.74E−03 | 2.29E−03 | 7.02E−03 | 7.02 | 2.6 |
| Muscle | 0.00E+00 | 1.24E−05 | 8.14E−03 | 8.15E−03 | 8.15 | 3.0 |
| Ovaries | 0.00E+00 | 0.00E+00 | 4.45E−03 | 4.45E−03 | 4.45 | 1.6 |
| Pancreas | 0.00E+00 | 9.05E−03 | 1.36E−02 | 2.27E−02 | 22.7 | 8.4 |
| Red Marrow | 0.00E+00 | 8.63E−05 | 6.37E−03 | 6.45E−03 | 6.45 | 2.4 |
| Osteogenic | 0.00E+00 | 3.17E−04 | 8.90E−03 | 9.22E−03 | 9.22 | 3.4 |
| Cells | | | | | | |
| Skin | 0.00E+00 | 0.00E+00 | 6.68E−03 | 6.68E−03 | 6.68 | 2.5 |
| Spleen | 0.00E+00 | 1.39E−02 | 1.75E−02 | 3.14E−02 | 31.4 | 11.6 |
| Tumor | 0.00E+00 | 3.75E+01 | 1.21E+00 | 3.87E+01 | 38700 | 14319.0 |
| Thymus | 0.00E+00 | 0.00E+00 | 7.57E−04 | 7.57E−04 | 0.757 | 0.3 |
| Thyroid | 0.00E+00 | 0.00E+00 | 1.38E−04 | 1.38E−04 | 0.138 | 0.1 |
| Urinary | 0.00E+00 | 0.00E+00 | 2.17E−02 | 2.17E−02 | 21.7 | 8.0 |
| Bladder | | | | | | |
| Wall | | | | | | |
| Uterus | 0.00E+00 | 0.00E+00 | 2.80E−03 | 2.80E−03 | 2.8 | 1.0 |
| Total | 0.00E+00 | 3.04E−02 | 8.79E−03 | 3.92E−02 | 39.2 | 14.5 |
| Body | | | | | | |

The value of ⁶⁸Ga-labeled small molecules targeting PSMA for imaging and staging primary prostate cancer and recurrent disease is becoming well established. Nevertheless, the short half-life (t_{1/2}=68 min) of this radioisotope precludes quantitation of radioligand distribution beyond a few hours post injection. Consequently, gallium-68 is a poor fit for pre-therapy dosimetry estimates prior to targeted radioligand therapy with lutetium-177 (t_{1/2}=6.65 d). To quantitate radioligand distribution and estimate absorbed dose, therefore, dosimetry studies may be performed with In-111 (t_{1/2}=2.81 d) as a surrogate for Lu-177, or with a low dose of Lu-177 itself. These studies may increase the number of administrations of radioactive material to patients and might also suffer from reduced imaging accuracy relative to PET imaging with Ga-68. In addition, the distribution of the ⁶⁸Ga—, ¹¹¹In—, and ¹⁷⁷Lu-labeled radioligands might vary even when the delivery vector is the same. In this light, the use of “sister” radioisotopes for such as Y-86/Y-90, Sc-44/Sc-47, and Cu-64/Cu-67 for quantitative imaging and therapy is appealing. The physical properties of copper-64 and copper-67, including well-matched half-lives, emission of short range particles with ideal energies for PET imaging and therapy, and the absence of coincidence photon emissions (Table 3), are ideal for theranostic use. Furthermore, large activities of these two radionuclides can be obtained in high radionuclidic purity. This facilitates the supply of ^{64/67}Cu-labeled radioligands to centers located at large distance from production sites.

TABLE 3
Decay properties of theranostic radionuclide pairs.
Positron Emitting Radionuclides for PSMA Theranostics

| Radionuclide | t_{1/2} | E(β⁺)ₘₑₐₙ (keV) | I (%) |
|---|---|---|---|
| Ga-68 | 67.8 min | 830 | 88.9 |
| Sc-44 | 3.97 h | 632 | 94.3 |
| Cu-64 | 12.7 h | 278 | 17.9 |
| Y-86 | 14.7 h | 550-898 | 31.9 |

β⁻ Emitting Radionuclides for PSMA Theranostics

| Radionuclide | t_{1/2} | E(β⁻)ₘₑₐₙ (keV) | I (%) |
|---|---|---|---|
| Lu-177 | 6.65 d | 134 | 100 |
| Sc-47 | 3.35 d | 162 | 100 |
| Cu-67 | 2.58 d | 141 | 100 |
| Y-90 | 2.67 d | 937 | 100 |

To fully exploit the potential of Cu-64/Cu-67 theranostics, efficient and stable chelation of the radiometal is required. In contrast to ⁶⁴Cu(II)-DOTA complexes, which are unstable in vivo, possibly as a consequence of the reduction of ⁶⁴Cu(II) to ⁶⁴Cu(I), cross-bridged macrocyclic chelators exhibit greater complex stability. Bifunctionalized 3,6,10,13,16,19-hexaazabicyclo[6.6.6]icosane, known as sarcophagine, chelators have been shown to form highly stable complexes with ⁶⁴Cu²⁺ when conjugated to peptides or peptide dimers. We confirm that the MeCOSar chelator allows low concentrations (<10 μM) of the small molecule RPS-085 to be labeled rapidly and efficiently with either copper-64 or copper-67 under mild conditions. The ability to quantitatively complex Cu²⁺ even in ligand concentrations of 10⁻⁷ M is a major advantage of MeCOSar over other commercially available chelators that can translate to substantial increases in the molar activities of radiolabeled compounds. [^{64/67}Cu]Cu-RPS-085 is stable in solution at room temperature for more than 24 h. This supports its centralized production and distribution to centers that do not have on-site ⁶⁸Ge/⁶⁸Ga generators. The radioligand is stable in human plasma at 37° C. beyond 24 h, and the absence of radioactivity in the mouse liver, the primary organ in which [^{64/67}Cu]Cu²⁺ accumulates, provides further evidence of the complex stability in vivo.

A challenging aspect of theranostic ligand development is the need to balance optimal pharmacokinetics for imaging, such as rapid tissue distribution and clearance from blood, with optimal characteristics for therapy, such as progressive and sustained tumor loading with concurrent clearance from normal tissue. This challenge is evident in the pharmacokinetics of previously described low molecular weight ⁶⁴Cu-labeled PSMA inhibitors: a phosphoramidate ligand with rapid clearance from blood and kidneys also exhibited low uptake in LNCaP xenograft tumors, with nearly complete washout by 48 h p.i., a trend that was also observed for urea-based ligands in PC3-PIP xenograft tumors. This latter family of ligands are most suitable for PET imaging at late time points. [⁶⁴Cu]Cu-RPS-085 displays excellent imaging characteristics even at early time points, including high tumor uptake and rapid clearance from background, leading to high tumor-to-background ratios by 4 h p.i. Furthermore, as activity in the tumor remains stable for as long as 48 h p.i., imaging at later time points, i.e. 24 h, could be performed to exploit increasing contrast to background without increasing the radiation exposure of the patient. Such a concept has already been validated clinically by [⁶⁴Cu]Cu-PSMA-617, which enabled high quality PET images to be obtained beyond 17 h post injection.

A second challenging aspect of small molecule ligand design is the inability to accurately and comprehensively predict the full impact of structural changes on compound pharmacokinetics. Previously, we and others have demonstrated that varying the length of the linker conjugating the PSMA binding group, the metal chelating moiety, and the albumin binding group influences both tumor uptake and retention and kidney clearance and retention.

In addition, varying albumin binding groups on a fixed linker structure also profoundly influences clearance from blood. Here we demonstrate that variation of the metal chelating moiety on a fixed structural platform also influences the pharmacokinetics of the ligand even when the chelator is not predicted to contribute to either PSMA binding or albumin binding.

This finding was recently described on a more compact molecular scaffold. The chelator appears to be responsible for a 10-fold decrease in affinity for PSMA relative to the DOTA-containing analogue. Furthermore, the overall charges of the Cu²⁺-MeCOSar complex, +2, and the Lu³⁺-p-SCN-Bn-DOTA complex, −1, are different. Without being bound by theory, this change in charge distribution may be responsible for changes in cooperativity of binding to these protein targets and for enhancing changes in compound pharmacokinetics, particularly with respect to renal clearance and retention.

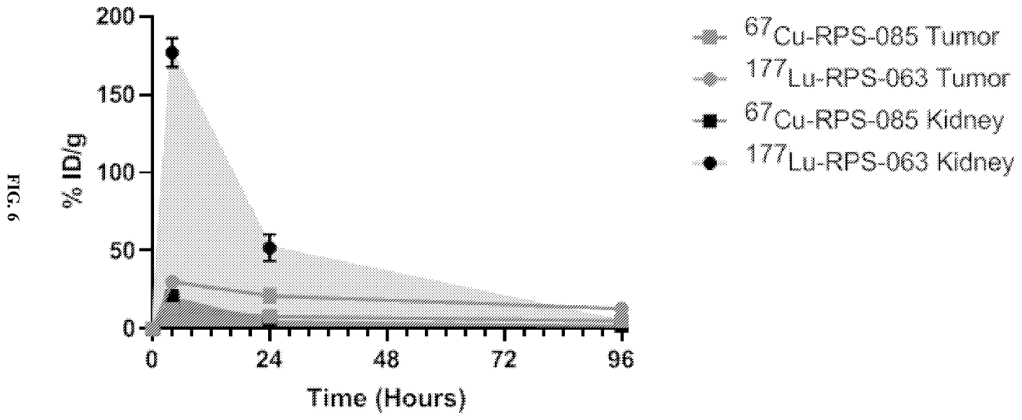

Affinity for serum albumin, and therefore activity in the blood, is lower than predicted on the basis of the structural similarity of RPS-085 to its previously reported cognate ligand. Consequently, we did not observe progressive tumor loading over time. However, [⁶⁷Cu]Cu-RPS-085 did not significantly clear from tumors over 24 h, leading to high accumulated activity (Ã). Although elimination from tumors is evident by 96 h p.i., Ã is 699 (% ID/g)·h. Concurrently, clearance from kidneys is rapid. The accumulated activity for the same time interval is 538 (% ID/g)·h (FIG. 6). Without being bound by theory, this is likely due to both accelerated plasma clearance and a small decrease in affinity for PSMA relative to other trifunctional ligands. By comparison, [¹⁷⁷Lu]Lu-RPS-063 achieves a substantially higher tumor integral of 1782 (% ID/g)·h in the same animal model. However, this is accompanied by A in kidneys of 4720 (% ID/g)·h (FIG. 6). These pharmacokinetics are reflected by the absorbed dose in tumors and kidneys. Over a 192 h period, the absorbed dose in tumors is approximately 16 times greater than kidneys for [⁶⁷Cu]Cu-RPS-085, but only 6.5 times greater for [¹⁷⁷Lu]Lu-RPS-063. Moreover, the rapid clearance of [⁶⁷Cu]Cu-RPS-085 leads to a 5-fold reduction in whole body absorbed dose relative to [¹⁷⁷Lu]Lu-RPS-063. We previously determined the accumulated activity in LNCaP tumors, Ãₜᵤₘₒᵣ, of [¹⁷⁷Lu]Lu-PSMA-617 to be 544 (% ID/g)·h over 96 h p.i., and activity in kidneys, Ã_{kidney}, to be 260 (% ID/g)·h in the same window [31]. Although the Ãₜᵤₘₒᵣ/Ã_{kidney} ratio of [⁶⁷Cu]Cu-RPS-085, 1.3, is slightly lower than that of [¹⁷⁷Lu]PSMA-617, 2.1, Ãₜᵤₘₒᵣ is 1.3 times greater. The therapeutic window of [⁶⁷Cu]Cu-RPS-085 may therefore be comparable to [¹⁷⁷Lu]Lu-PSMA-617 and much greater than [¹⁷⁷Lu]Lu-RPS-063.

Notwithstanding the promising characteristics of RPS-085 as a potential ligand for radioligand therapy, further gains could be achieved by extending plasma half-life. This approach has recently been employed to develop [⁶⁴Cu]Cu-PSMA-ALB-89, which demonstrates progressive accumulation in xenograft tumors over 24 h. However, the high kidney activity and non-negligible accumulation of activity in the liver may compromise the translation of this compound relative to RPS-085. We have recently explored extended polyethylene glycol (PEG) linkers as a method of modulating plasma pharmacokinetics, and achieved accelerated kidney clearance with sustained tumor activity. Incorporation of the bifunctionalized MeCOSar chelator into this molecular scaffold may further increase the dose delivered to tumors.

Uptake of numerous PSMA-targeting ligands in salivary and lacrimal glands has been reported, and toxicity in these structures due to emission of β⁻, and especially α-particles, is dose-limiting. The small size of murine salivary and lacrimal glands makes quantification of dose absorbed challenging by either imaging or biodistribution studies. Therefore we are unable to acutely predict the possible toxicity of [⁶⁷Cu]Cu-RPS-085 in these structures. However, the 3 particles emitted by copper-67 are of medium energy (141 keV), such that the predicted absorbed dose in normal tissue is similar to lutetium-177 and smaller than iodine-131. [⁶⁷Cu]Cu-RPS-085 clears from normal tissue at a similar rate to [¹⁷⁷Lu]Lu-PSMA-617 in the same xenograft mouse model. We therefore anticipate that toxicity to salivary glands should be, at a minimum, no worse than the ¹⁷⁷Lu-labeled PSMA ligands currently under clinical investigation.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary.

It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group.

Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A compound comprising
   a tumor targeting domain comprising a moiety capable of recognizing or interacting with a molecular target on the surface of tumor cells;
   a blood-protein binding domain; and
   a sarcophagine-containing domain; wherein the moiety of the tumor targeting domain is distal to and sterically unimpeded by the blood-protein binding domain.
B. The compound of Paragraph A, wherein the tumor targeting domain binds to a tumor associated molecular target selected from one or more of a tumor-specific cell surface protein, prostate specific membrane antigen (PSMA), somatostatin peptide receptor-2 (SSTR2), alphavbeta3 (αvβ3), alphavbeta6, a gastrin-releasing peptide receptor, a seprase, fibroblast activation protein alpha (FAP-alpha), an incretin receptor, a glucose-dependent insulinotropic polypeptide receptor, VIP-1, NPY, a folate receptor, LHRH, a neuronal transporter (e.g., noradrenaline transporter (NET)), EGFR, HER-2, VGFR, MUC-1, CEA, MUC-4, ED2, TF-antigen, an endothelial specific marker, neuropeptide Y, uPAR, TAG-72, a claudin, a CCK analog, VIP, bombesin, VEGFR, a tumor-specific cell surface protein, GLP-1, CXCR4, Hepsin, TMPRSS2, a caspace, cMET, or an overexpressed peptide receptor.
C. The compound of Paragraph A or Paragraph B, wherein the tumor targeting domain comprises a modified antibody, modified antibody fragment, a modified binding peptide, a prostate specific membrane antigen (“PSMA”) binding peptide, a somatostatin receptor agonist, a bombesin receptor agonist, a seprase binding compound, or binding fragment of any one or more thereof.
D. The compound of any one of Paragraphs A-C, wherein the tumor targeting domain comprises belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab.
E. The compound of any one of Paragraphs A-C, wherein the tumor targeting domain comprises an antigen-binding fragment of belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab.
F. The compound of any one of Paragraphs A-E, wherein the compound is of any one of Formulas I-V:

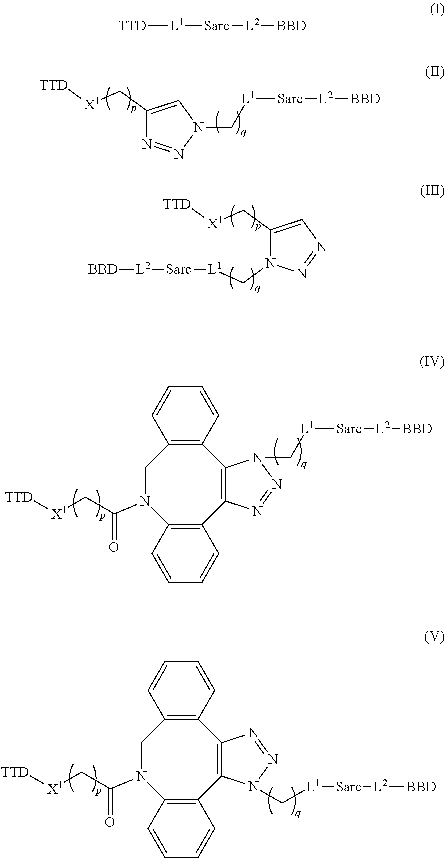

or a pharmaceutically acceptable salt and/or solvate thereof, wherein
   TTD is the tumor targeting domain;
   BBD is the blood-protein binding domain;
   Sarc is the sarcophagine-containing domain;
   X¹ is independently at each occurrence absent, O, S, NH, —C(O)—, —C(O)—NR¹—, —NR²—C(O)—, —C(O)—NR³—C₁-C₁₂ alkylene-, —C₁-C₁₂ alkylene-C(O)—, —C(O)—NR⁴—C₁-C₁₂ alkylene-C(O)—, -arylene-, -heterocyclene-, —O(CH₂CH₂O)ₐ—, —CH₂CH₂—O(CH₂CH₂O)_{b}—, —CH₂CH₂—O(CH₂CH₂O)_{c}—CH₂CH₂—, —O(CH₂CH₂O)_{d}—CH₂CH₂—, —C(O)—O(CH₂CH₂O)ₑ—, —O(CH₂CH₂O)_{f}—CH₂CH₂C(O)—, —C(O)—O(CH₂CH₂O)_{g}—, —C(O)—O(CH₂CH₂O)ₕ—CH₂CH₂—, —C(O)—O(CH₂CH₂O)ᵢ—CH₂CH₂C(O)—, —CH₂CH₂—O(CH₂CH₂O)ⱼ—CH₂CH₂C(O)—, —C(O)—NR⁵—CH₂CH₂O(CH₂CH₂O)ₖ—, —C(O)—NR⁶—CH₂CH₂O(CH₂CH₂O)ₗ—CH₂CH₂—, —C(O)—NR⁷—CH₂CH₂O(CH₂CH₂O)ₘ—CH₂CH₂C(O)—, an amino acid, a peptide of 2, 3, 4, 5, 6, 7, 8, 9, or 10 amino acids, or a combination of any two or more thereof, where a, b, c, d, e, f, g, h, i, j, k, l, and m are independently at each occurrence 0, 1, 2, 3, 4, 5, 6, 7, 8, 9, 10, 11, 12, 13, 14, 15, 16, 17, 18, or 19, and where R¹, R², R³, R⁴, R⁵, R⁶, and R⁷ are independently at each occurrence H, alkyl, or aryl;
   L¹ is independently at each occurrence absent, O, S, NH, —C(O)—, —C(O)—NR⁸—, —NR⁹—C(O)—, —C(O)—NR¹⁰—C₁-C₁₂ alkylene-, —C₁-C₁₂ alkylene-C(O)—, —C(O)—NR¹¹—C₁-C₁₂ alkylene-C(O)—, -arylene-, -heterocyclene-, —O(CH₂CH₂O)_{a′}—, —CH₂CH₂—O(CH₂CH₂O)_{b′}—, —CH₂CH₂—O(CH₂CH₂O)_{c′}—CH₂CH₂—, —O(CH₂CH₂O)_{d′}—CH₂CH₂—, —C(O)—O(CH₂CH₂O)_{e′}—, —O(CH₂CH₂O)_{f′}—CH₂CH₂C(O)—, —C(O)—O(CH₂CH₂O)_{g′}—, —C(O)—O(CH₂CH₂O)_{h′}—CH₂CH₂—, —C(O)—O(CH₂CH₂O)_{i′}—CH₂CH₂C(O)—, —CH₂CH₂—O(CH₂CH₂O)_{j′}—CH₂CH₂C(O)—, —C(O)—NR²—CH₂CH₂O(CH₂CH₂O)_{k′}—, —C(O)—NR¹³—CH₂CH₂O(CH₂CH₂O)_{l′}—CH₂CH₂—, —C(O)—NR¹⁴—CH₂CH₂O(CH₂CH₂O)_{m′}—CH₂CH₂C(O)—, an amino acid, a peptide of 2, 3, 4, 5, 6, 7, 8, 9, or 10 amino acids, or a combination of any two or more thereof, where a′, b′, c′, d′, e′, f′, g′, h′, i′, j′, k′, l′, and m′ are independently at each occurrence 0, 1, 2, 3, 4, 5, 6, 7, 8, 9, 10, 11, 12, 13, 14, 15, 16, 17, 18, or 19, and where R⁸, R⁹, R¹⁰, R¹¹, R¹², R¹³, and R¹⁴ are independently at each occurrence H, alkyl, or aryl;
   L² is independently at each occurrence absent, O, S, NH, —C(O)—, —C(O)—NR¹⁵—, —NR¹⁶—C(O)—, —C(O)—NR¹⁷—C₁-C₁₂ alkylene-, —C₁-C₁₂ alkylene-C(O)—, —C(O)—NR¹⁸—C₁-C₁₂ alkylene-C(O)—, -arylene-, -heterocyclene-, —O(CH₂CH₂O)_{a″}—, —CH₂CH₂—O(CH₂CH₂O)_{b″}—, —CH₂CH₂—O(CH₂CH₂O)_{c″}, —CH₂CH₂—, —O(CH₂CH₂O)_{d″}—CH₂CH₂—, —C(O)—O(CH₂CH₂O)_{e″}—, —O(CH₂CH₂O)_{f″}—CH₂CH₂C(O)—, —C(O)—O(CH₂CH₂O)_{g″}—, —C(O)—O(CH₂CH₂O)_{h″}—CH₂CH₂—, —C(O)—O(CH₂CH₂O)_{i″}—CH₂CH₂C(O)—, —CH₂CH₂—O(CH₂CH₂O)_{j″}, —CH₂CH₂C(O)—, —C(O)—NR¹⁹—CH₂CH₂O(CH₂CH₂O)_{k″}—, —C(O)—NR²⁰—CH₂CH₂O(CH₂CH₂O)_{l″}—CH₂CH₂—, —C(O)—NR²¹—CH₂CH₂O(CH₂CH₂O)_{m″}—CH₂CH₂C(O)—, an amino acid, a peptide of 2, 3, 4, 5, 6, 7, 8, 9, or 10 amino acids, or a combination of any two or more thereof, where a″, b″, c″, d″, e″, f″, g″, h″, i″, j″, k″, l″, and m″ are independently at each occurrence 0, 1, 2, 3, 4, 5, 6, 7, 8, 9, 10, 11, 12, 13, 14, 15, 16, 17, 18, or 19, and where R¹⁵, R¹⁶, R¹⁷, R¹⁸, R¹⁹, R²⁰, and R²¹ are independently at each occurrence H, alkyl, or aryl;
   p is independently at each occurrence 0, 1, 2, 3, 4, or 5; and
   q is independently at each occurrence 1 or 2.
G. The compound of any one of Paragraphs A-F, wherein the tumor targeting domain is

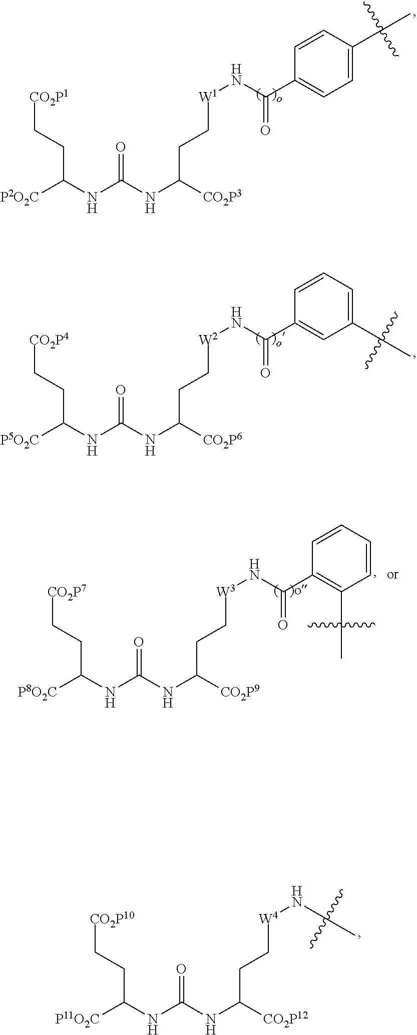

wherein
W¹, W², W³, and W⁴ are each independently —C(O)—, —(CH₂)ᵣ—, or —(CH₂)ₛNH—C(O)—;
r is independently at each occurrence 1 or 2;
s is independently at each occurrence 1 or 2;
P¹, P², P³, P⁴, P⁵, P⁶, P⁷, P⁸, P⁹, P¹⁰, P¹¹, and P¹² are each independently H, methyl, benzyl, 4-methoxybenzyl, or tert-butyl; and
o, o′, and o″ are each independently 0 or 1.
H. The compound of Paragraph G, wherein P¹, P², P³, P⁴, P⁵, P⁶, P⁷, P⁸, P⁹, P¹⁰, P¹¹, and P¹² are each independently H or tert-butyl.
I. The compound of Paragraph G or Paragraph H, wherein P¹, P², P³, P⁴, P⁵, P⁶, P⁷, P⁸, P⁹, P¹⁰, P¹¹, and P¹² are each independently H.
J. The compound of any one of Paragraphs A-I, wherein the blood-protein binding domain is

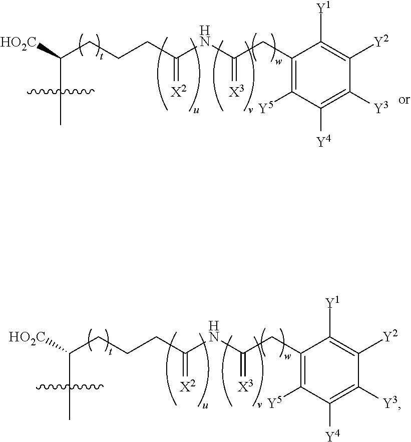

wherein
Y¹, Y², Y³, Y⁴, and Y⁵ are independently at each occurrence H, halo, or alkyl;
X² and X³ are each independently O or S
t is independently at each occurrence 0, 1, or 2;
u is independently at each occurrence 0 or 1;
v is independently at each occurrence 0 or 1; and
w is independently at each occurrence 0, 1, 2, 3, or 4, optionally wherein u and v cannot be the same value.
K. The compound of any one of Paragraphs A-I, wherein the blood-protein binding domain comprises myristic acid, a substituted or unsubstituted indole-2-carboxylic acid, a substituted or unsubstituted thioamide, a substituted or unsubstituted 4-oxo-4-(5,6,7,8-tetrahydronaphthalen-2-yl)butanoic acid, a substituted or unsubstituted naphthalene acylsulfonamide, a substituted or unsubstituted diphenylcyclohexanol phosphate ester, a substituted or unsubstituted 4-iodophenylalkanoic acid, a substituted or unsubstituted 3-(4-iodophenyl)propionic acid, a substituted or unsubstituted 2-(4-iodophenyl)acetic acid, or a substituted or unsubstituted 4-(4-iodophenyl)butanoic acid.
L. The compound of any one of Paragraphs A-I, wherein the blood-protein binding domain is

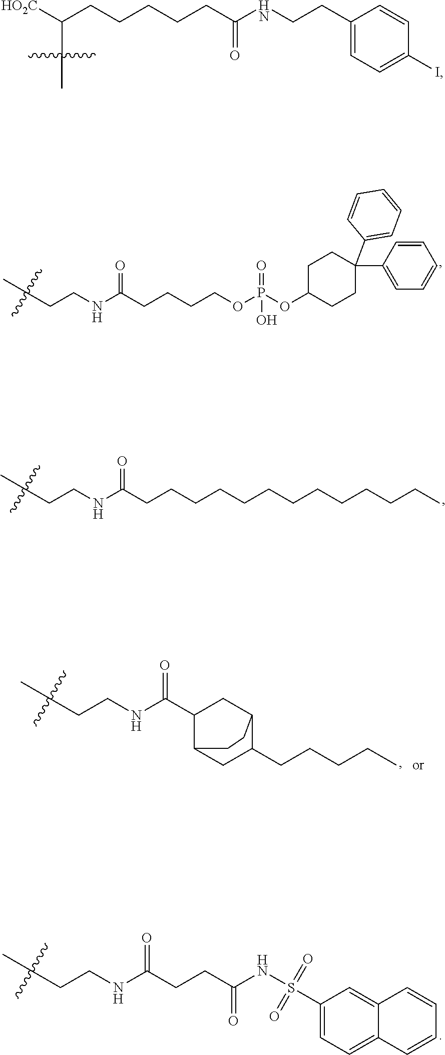 [SMILES: *C(CCCCCC(=O)NCCc1ccc(I)cc1)C(=O)O ; *CCNC(=O)CCCCOP(=O)(O)OC1CCC(c2ccccc2)(c2ccccc2)CC1 ; *CCNC(=O)CCCCCCCCCCCCC ; *CCNC(=O)C1CC2CCC1CC2CCCCC ; *CCNC(=O)CCC(=O)NS(=O)(=O)c1ccc2ccccc2c1]

M. The compound of any one of Paragraphs A-L, wherein the sarcophagine-containing domain is

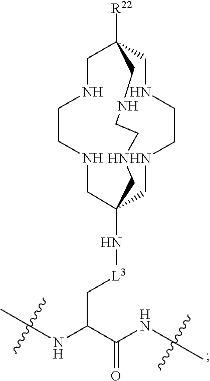

wherein
R²² is H, alkyl, aryl, or NR²³R²⁴;
R²³ and R²⁴ are each independently H, alkyl, aryl, alkanoyl, or aryloyl;
L³ is absent, —C(O)—, —C₁-C₁₂ alkylene-, —C₁-C₁₂ alkylene-C(O)—, —NR²⁵C(O)—C₁-C₁₂ alkylene-C(O)—, —C₁-C₁₂ alkylene-NR²⁵C(O)—C₁-C₁₂ alkylene-C(O)—, -arylene-, —C₁-C₁₂ alkylene-C(O)NR²⁵—CH₂-phenylene-CH₂—, —C₁-C₁₂ alkylene-C(O)NR²⁵—CH₂-phenylene-C(O)—, —C₁-C₁₂ alkylene-NR²⁵C(O)—C₁-C₁₂ alkylene-C(O)—C(O)NR²⁵—CH₂-phenylene-CH₂—, or —C₁-C₁₂ alkylene-NR²⁵C(O)—C₁-C₁₂ alkylene-C(O)—C(O)NR²⁵—CH₂-phenylene-C(O)—; and
R²⁵ is independently at each occurrence H, alkyl, or aryl.
N. The compound of Paragraph M, wherein R²² is H, methyl, or NH₂.
O. The compound of any one of Paragraphs A-N, wherein the sarcophagine-containing domain chelates ⁶⁴Cu⁺² or ⁶⁷Cu⁺².
P. A composition comprising a compound of any one of Paragraphs A-O and a pharmaceutically acceptable carrier.
Q. A pharmaceutical composition, the composition comprising
   an effective amount of a compound of Paragraph O for imaging and/or detecting one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer; and
   a pharmaceutically acceptable carrier.
R. The pharmaceutical composition of any one of Paragraph Q, wherein the pharmaceutical composition is formulated for intravenous administration, optionally comprising sterilized water, Ringer's solution, or an isotonic aqueous saline solution.
S. The pharmaceutical composition of Paragraph Q or Paragraph R, wherein the effective amount of the compound is from about 0.01 μg to about 10 mg of the compound per gram of the pharmaceutical composition.
T. The pharmaceutical composition of any one of Paragraphs Q-S, wherein the pharmaceutical composition is provided in an injectable dosage form.
U. A method comprising
   administering to a subject an effective amount of a compound of Paragraph O for imaging and/or detecting a cancer; and
   subsequent to the administering, detecting one or more of positron emission, gamma rays from positron emission and annihilation, and Cerenkov radiation due to positron emission.
V. The method of Paragraph U, wherein the cancer comprises one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer.
W. The method of Paragraph U or Paragraph V, wherein the subject is suspected of suffering from a mammalian tissue overexpressing prostate specific membrane antigen (“PSMA”).
X. The method of any one of Paragraphs U-W, wherein the mammalian tissue comprises one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer.
Y. The method of Paragraph X, wherein administering the compound comprises parenteral administration or intravenous administration.
Z. A pharmaceutical composition comprising
   an effective amount of a compound of Paragraph O for treating one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer; and
   a pharmaceutically acceptable carrier.
AA. The pharmaceutical composition of Paragraph Z, wherein the pharmaceutical composition is formulated for intravenous administration, optionally comprising sterilized water, Ringer's solution, or an isotonic aqueous saline solution.
AB. The pharmaceutical composition of Paragraph Z or Paragraph AA, wherein the effective amount of the compound is from about 0.01 μg to about 10 mg of the compound per gram of the pharmaceutical composition.
AC. The pharmaceutical composition of any one of Paragraphs Z-AB, wherein the pharmaceutical composition is provided in an injectable dosage form.
AD. The pharmaceutical composition of any one of Paragraphs Z-AC, wherein the effective amount of the compound for treating one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer is also an effective amount of the compound for imaging and/or detecting one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer.
AE. A method comprising
   administering to a subject an effective amount of a compound of Paragraph O for treating a cancer.
AF. The method of Paragraph AE, wherein the cancer comprises one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer.
AG. The method of Paragraph AE or Paragraph AF, wherein administering the compound comprises parenteral administration.
AH. The method of any one of Paragraphs AE-AG, wherein administering the compound comprises intravenous administration.
AI. The method of any one of Paragraphs AE-AH, wherein the effective amount of the compound for treating the cancer is from about 0.1 μg to about 50 μg per kilogram of subject mass.
AJ. The method of any one of Paragraphs AE-AI, wherein the effective amount of the compound is also an effective amount of the compound for imaging and/or detecting the cancer.

Other embodiments are set forth in the following claims, along with the full scope of equivalents to which such claims are entitled.

1. A compound comprising
a tumor targeting domain comprising a moiety capable of recognizing or interacting with a molecular target on the surface of tumor cells;
a blood-protein binding domain; and
a sarcophagine-containing domain; wherein the moiety of the tumor targeting domain is distal to and sterically unimpeded by the blood-protein binding domain.

2. The compound of claim 1, wherein the tumor targeting domain binds to a tumor associated molecular target selected from one or more of a tumor-specific cell surface protein, prostate specific membrane antigen (PSMA), somatostatin peptide receptor-2 (SSTR2), alphavbeta3 (αvβ3), alphavbeta6, a gastrin-releasing peptide receptor, a seprase, fibroblast activation protein alpha (FAP-alpha), an incretin receptor, a glucose-dependent insulinotropic polypeptide receptor, VIP-1, NPY, a folate receptor, LHRH, a neuronal transporter (e.g., noradrenaline transporter (NET)), EGFR, HER-2, VGFR, MUC-1, CEA, MUC-4, ED2, TF-antigen, an endothelial specific marker, neuropeptide Y, uPAR, TAG-72, a claudin, a CCK analog, VIP, bombesin, VEGFR, a tumor-specific cell surface protein, GLP-1, CXCR4, Hepsin, TMPRSS2, a caspace, cMET, or an overexpressed peptide receptor.

3. The compound of claim 1, wherein the tumor targeting domain comprises a modified antibody, modified antibody fragment, a modified binding peptide, a prostate specific membrane antigen (“PSMA”) binding peptide, a somatostatin receptor agonist, a bombesin receptor agonist, a seprase binding compound, or binding fragment of any one or more thereof.

4. The compound of claim 1, wherein the tumor targeting domain comprises belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab.

5. The compound of claim 1, wherein the tumor targeting domain comprises an antigen-binding fragment of belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab.

6. The compound of claim 1, wherein the compound is of any one of Formulas I-V: 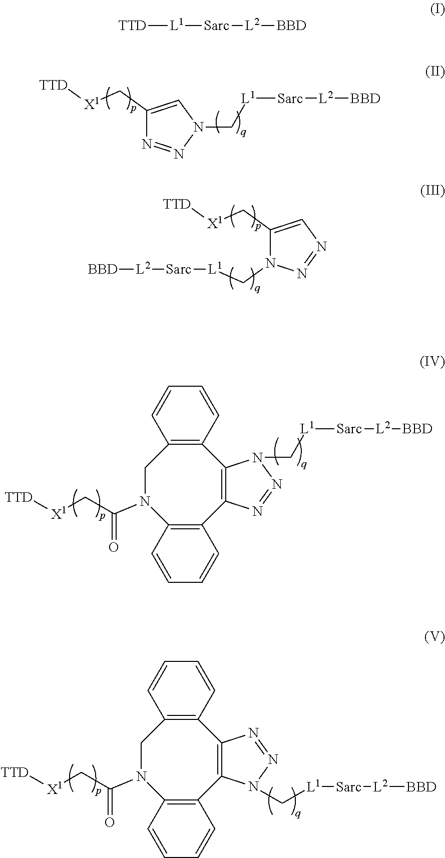
or a pharmaceutically acceptable salt and/or solvate thereof, wherein
TTD is the tumor targeting domain;
BBD is the blood-protein binding domain;
Sarc is the sarcophagine-containing domain;
X¹ is independently at each occurrence absent, O, S, NH, —C(O)—, —C(O)—NR¹—, —NR²—C(O)—, —C(O)—NR³—C₁-C₁₂ alkylene-, —C₁-C₁₂ alkylene-C(O)—, —C(O)—NR⁴—C₁-C₁₂ alkylene-C(O)—, -arylene-, -heterocyclene-, —O(CH₂CH₂O)ₐ—, —CH₂CH₂—O(CH₂CH₂O)_{b}—, —CH₂CH₂—O(CH₂CH₂O)_{c}—CH₂CH₂—, —O(CH₂CH₂O)_{d}—CH₂CH₂—C(O)—O(CH₂CH₂O)ₑ—, —O(CH₂CH₂O)_{f}—CH₂CH₂C(O)—, —C(O)—O(CH₂CH₂O)_{g}—C(O)—O(CH₂CH₂O)ₕ—CH₂CH₂—, —C(O)—O(CH₂CH₂O)ᵢ—CH₂CH₂C(O)—, —CH₂CH₂—O(CH₂CH₂O)ⱼ—CH₂CH₂C(O)—, —C(O)—NR⁵—CH₂CH₂O(CH₂CH₂O)ₖ—, —C(O)—NR⁶—CH₂CH₂O(CH₂CH₂O)ₗ—CH₂CH₂—, —C(O)—NR⁷—CH₂CH₂O(CH₂CH₂O)ₘ—CH₂CH₂C(O)—, an amino acid, a peptide of 2, 3, 4, 5, 6, 7, 8, 9, or 10 amino acids, or a combination of any two or more thereof, where a, b, c, d, e, f, g, h, i, j, k, l, and m are independently at each occurrence 0, 1, 2, 3, 4, 5, 6, 7, 8, 9, 10, 11, 12, 13, 14, 15, 16, 17, 18, or 19, and where R¹, R², R³, R⁴, R⁵, R⁶, and R⁷ are independently at each occurrence H, alkyl, or aryl;
L¹ is independently at each occurrence absent, O, S, NH, —C(O)—, —C(O)—NR⁸—, —NR⁹—C(O)—, —C(O)—NR¹⁰—C₁-C₁₂ alkylene-, —C₁-C₁₂ alkylene-C(O)—, —C(O)—NR¹¹—C₁-C₁₂ alkylene-C(O)—, -arylene-, -heterocyclene-, —O(CH₂CH₂O)_{a′}—, —CH₂CH₂—O(CH₂CH₂O)_{b′}—, —CH₂CH₂—O(CH₂CH₂O)_{c′}—CH₂CH₂—, —O(CH₂CH₂O)_{d′}—CH₂CH₂—, —C(O)—O(CH₂CH₂O)_{e′}—, —O(CH₂CH₂O)_{f′}—CH₂CH₂C(O)—, —C(O)—O(CH₂CH₂O)_{g′}—, —C(O)—O(CH₂CH₂O)_{h′}—CH₂CH₂—, —C(O)—O(CH₂CH₂O)_{i′}—CH₂CH₂C(O)—, —CH₂CH₂—O(CH₂CH₂O)_{j′}—CH₂CH₂C(O)—, —C(O)—NR¹²—CH₂CH₂O(CH₂CH₂O)_{k′}—, —C(O)—NR¹³—CH₂CH₂O(CH₂CH₂O)_{l′}—CH₂CH₂—, —C(O)—NR¹⁴—CH₂CH₂O(CH₂CH₂O)_{m′}—CH₂CH₂C(O)—, an amino acid, a peptide of 2, 3, 4, 5, 6, 7, 8, 9, or 10 amino acids, or a combination of any two or more thereof, where a′, b′, c′, d′, e′, f′, g′, h′, i′, j′, k′, l′, and m′ are independently at each occurrence 0, 1, 2, 3, 4, 5, 6, 7, 8, 9, 10, 11, 12, 13, 14, 15, 16, 17, 18, or 19, and where R⁸, R⁹, R¹⁰, R¹¹, R¹², R¹³, and R¹⁴ are independently at each occurrence H, alkyl, or aryl;
L² is independently at each occurrence absent, O, S, NH, —C(O)—, —C(O)—NR¹⁵-, —NR¹⁶—C(O)—, —C(O)—NR¹⁷—C₁-C₁₂ alkylene-, —C₁-C₁₂ alkylene-C(O)—, —C(O)—NR¹⁸—C₁-C₁₂ alkylene-C(O)—, -arylene-, -heterocyclene-, —O(CH₂CH₂O)_{a″}—, —CH₂CH₂—O(CH₂CH₂O)_{b″}—, —CH₂CH₂—O(CH₂CH₂O)_{c″}, —CH₂CH₂—, —O(CH₂CH₂O)_{d″}—CH₂CH₂—, —C(O)—O(CH₂CH₂O)_{e″}—, —O(CH₂CH₂O)_{f″}—CH₂CH₂C(O)—, —C(O)—O(CH₂CH₂O)_{g″}—, —C(O)—O(CH₂CH₂O)_{h″}—CH₂CH₂—, —C(O)—O(CH₂CH₂O)_{i″}—CH₂CH₂C(O)—, —CH₂CH₂—O(CH₂CH₂O)_{j″}—CH₂CH₂C(O)—, —C(O)—NR¹⁹—CH₂CH₂O(CH₂CH₂O)_{k″}—, —C(O)—NR²⁰—CH₂CH₂O(CH₂CH₂O)_{l″}—CH₂CH₂—, —C(O)—NR²¹—CH₂CH₂O(CH₂CH₂O)_{m″}—CH₂CH₂C(O)—, an amino acid, a peptide of 2, 3, 4, 5, 6, 7, 8, 9, or 10 amino acids, or a combination of any two or more thereof, where a″, b″, c″, d″, e″, f″, g″, h″, i″, j″, k″, l″, and m″ are independently at each occurrence 0, 1, 2, 3, 4, 5, 6, 7, 8, 9, 10, 11, 12, 13, 14, 15, 16, 17, 18, or 19, and where R¹⁵, R¹⁶, R¹⁷, R¹⁸, R¹⁹, R²⁰, and R²¹ are independently at each occurrence H, alkyl, or aryl;
p is independently at each occurrence 0, 1, 2, 3, 4, or 5; and
q is independently at each occurrence 1 or 2.

7. The compound of claim 1, wherein the tumor targeting domain is 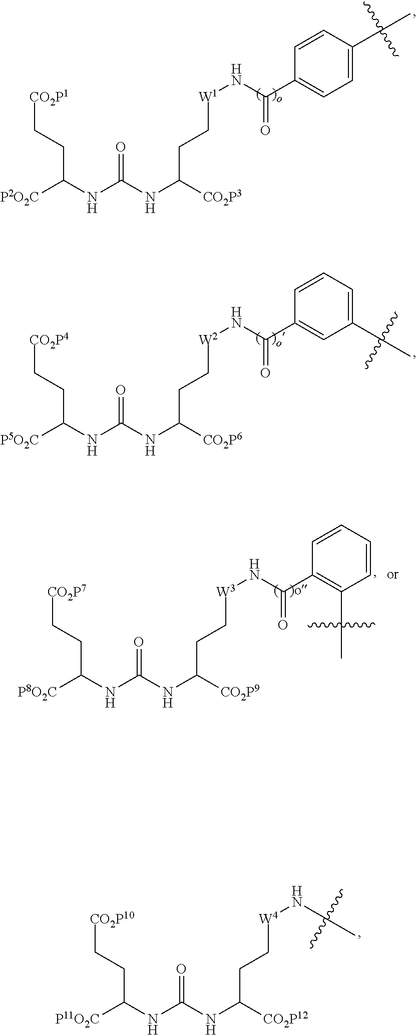
wherein
W¹, W², W³, and W⁴ are each independently —C(O)—, —(CH₂)ᵣ—, or —(CH₂)ₛ—NH—C(O)—;
r is independently at each occurrence 1 or 2;
s is independently at each occurrence 1 or 2;
P¹, P², P³, P⁴, P⁵, P⁶, P⁷, P⁸, P⁹, P¹⁰, P¹¹, and P¹² are each independently H, methyl, benzyl, 4-methoxybenzyl, or tert-butyl; and
o, o′, and o″ are each independently 0 or 1.

8. The compound of claim 7, wherein P¹, P², P³, P⁴, P⁵, P⁶, P⁷, P⁸, P⁹, P¹⁰, P¹¹, and P¹² are each independently H or tert-butyl.

9. The compound of claim 7, wherein P¹, P², P³, P⁴, P⁵, P⁶, P⁷, P⁸, P⁹, P¹⁰, P¹¹, and P¹² are each independently H.

10. The compound of claim 1, wherein the blood-protein binding domain is 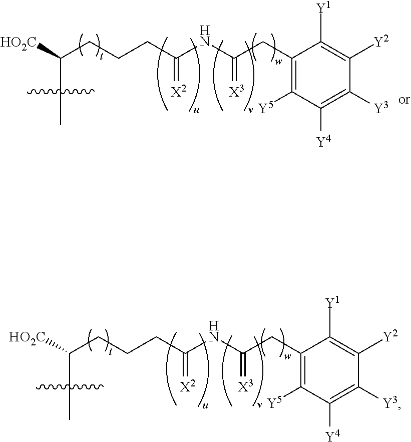
wherein
Y¹, Y², Y³, Y⁴, and Y⁵ are independently at each occurrence H, halo, or alkyl;
X² and X³ are each independently O or S
t is independently at each occurrence 0, 1, or 2;
u is independently at each occurrence 0 or 1;
v is independently at each occurrence 0 or 1; and
w is independently at each occurrence 0, 1, 2, 3, or 4, optionally wherein u and v cannot be the same value.

11. The compound of claim 1, wherein the blood-protein binding domain comprises myristic acid, a substituted or unsubstituted indole-2-carboxylic acid, a substituted or unsubstituted thioamide, a substituted or unsubstituted 4-oxo-4-(5,6,7,8-tetrahydronaphthalen-2-yl)butanoic acid, a substituted or unsubstituted naphthalene acylsulfonamide, a substituted or unsubstituted diphenylcyclohexanol phosphate ester, a substituted or unsubstituted 4-iodophenylalkanoic acid, a substituted or unsubstituted 3-(4-iodophenyl)propionic acid, a substituted or unsubstituted 2-(4-iodophenyl)acetic acid, or a substituted or unsubstituted 4-(4-iodophenyl)butanoic acid.

12. The compound of claim 1, wherein the blood-protein binding domain is

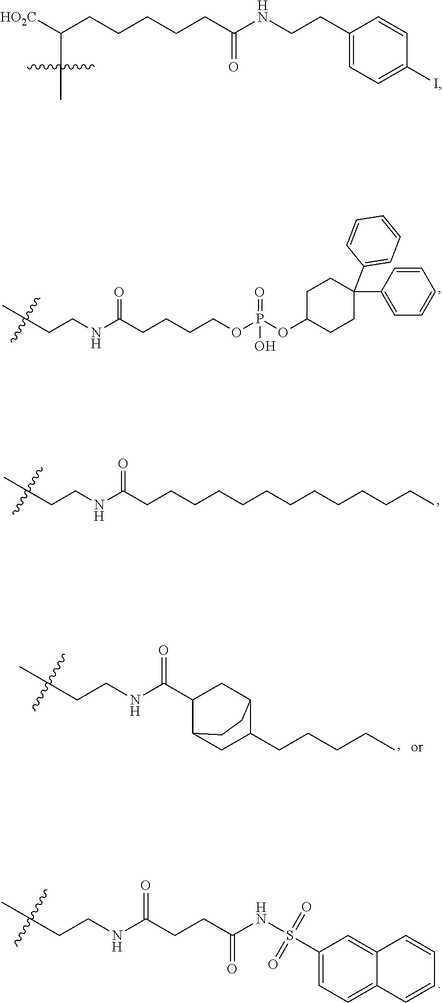 [SMILES: *C(CCCCCC(=O)NCCc1ccc(I)cc1)C(=O)O ; *CCNC(=O)CCCCOP(=O)(O)OC1CCC(c2ccccc2)(c2ccccc2)CC1 ; *CCNC(=O)CCCCCCCCCCCCC ; *CCNC(=O)C1CC2CCC1CC2CCCCC ; *CCNC(=O)CCC(=O)NS(=O)(=O)c1ccc2ccccc2c1]

13. The compound of claim 1, wherein the sarcophagine-containing domain is 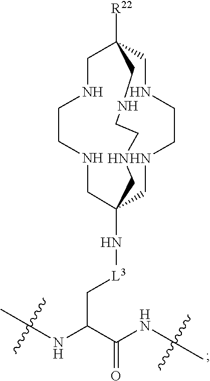
wherein
R²² is H, alkyl, aryl, or NR²³R24
R²³ and R²⁴ are each independently H, alkyl, aryl, alkanoyl, or aryloyl;
L³ is absent, —C(O)—, —C₁-C₁₂ alkylene-, —C₁-C₁₂ alkylene-C(O)—, —NR²⁵C(O)—C₁-C₁₂ alkylene-C(O)—, —C₁-C₁₂ alkylene-NR²⁵C(O)—C₁-C₁₂ alkylene-C(O)—, -arylene-, —C₁-C₁₂ alkylene-C(O)NR²⁵—CH₂-phenylene-CH₂—, —C₁-C₁₂ alkylene-C(O)NR²⁵—CH₂-phenylene-C(O)—, —C₁-C₁₂ alkylene-NR²⁵C(O)—C₁-C₁₂ alkylene-C(O)—C(O)NR²⁵—CH₂-phenylene-CH₂—, or —C₁-C₁₂ alkylene-NR²⁵C(O)—C₁-C₁₂ alkylene-C(O)—C(O)NR²⁵—CH₂-phenylene-C(O)—; and
R²⁵ is independently at each occurrence H, alkyl, or aryl.

14. The compound of claim 13, wherein R²² is H, methyl, or NH₂.

15. The compound of claim 1, wherein the sarcophagine-containing domain chelates ⁶⁴Cu⁺² or ⁶⁷Cu⁺².

16. A composition comprising a compound of claim 1 and a pharmaceutically acceptable carrier.

17. A pharmaceutical composition, the composition comprising
an effective amount of a compound of claim 15 for imaging and/or detecting one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer; and
a pharmaceutically acceptable carrier.

18. A method comprising
administering to a subject an effective amount of a compound of claim 15 for imaging and/or detecting a cancer; and
subsequent to the administering, detecting one or more of positron emission, gamma rays from positron emission and annihilation, and Cerenkov radiation due to positron emission.

19.-22. (canceled)

23. A pharmaceutical composition comprising
an effective amount of a compound of claim 15 for treating one or more of non-small cell lung cancer, small cell carcinoma of the lung, bladder cancer, colon cancer, gallbladder cancer, pancreatic cancer, esophageal cancer, melanoma, liver cancer, primary gastric adenocarcinoma, primary colorectal adenocarcinoma, renal cell carcinoma, prostate cancer, a neuroendocrine tumor, a pituitary tumor, a vasoactive intestinal peptide-secreting tumor, a glioma, breast cancer, an adrenal cortical cancer, a cervical carcinoma, a vulvar carcinoma, an endometrial carcinoma, a primary ovarian carcinoma, a metastatic ovarian carcinoma, and a metastatic cancer; and
a pharmaceutically acceptable carrier.

24. A method comprising
administering to a subject an effective amount of a compound of claim 15 for treating a cancer.

25.-26. (canceled)
